(19) United States
(12) Patent Application Publication
(10) Pub. No.: US 2020/0056203 A1
(43) Pub. Date: Feb. 20, 2020

(54) Treating Animal Cancers Through Programmed Cancer Cell Death

(71) Applicant: Richard Postrel, Miami Beach, FL (US)

(72) Inventor: Richard Postrel, Miami Beach, FL (US)

(21) Appl. No.: 16/104,161

(22) Filed: Aug. 17, 2018

(51) Int. Cl. C12N 15/86; A61K 35/76; C07K 14/74; A61K 38/17; A61K 31/59; C07K 14/005; A61K 38/16; A61K 45/06

(52) U.S. Cl. CPC C12N 15/86; A61K 35/76; C07K 14/70539; A61K 38/1774; C12N 2760/16033; C07K 14/005; A61K 38/162; A61K 45/06; C12N 2760/16043; A61K 31/59

(57) ABSTRACT

The present invention provides systems and methods wherethrough a cancer cell's metabolic activities attract a vector to the cell to provide the cell with tools and instructions for self-destruction. A vector is engineered for attraction to and reaction with cells of higher temperature, a characteristic result of hypermetabolism inherent in the uncontrolled or extreme growth of cancerous and precancerous cells. A second engineered feature relates to the increased acidity resulting from a cancer cell's reduced reliance of mitochondrial ATP production. The engineered vector then stimulates the body's natural intracellular and extracellular innate immune responses to effect death and destruction of the targeted hypermetabolizing cells.

Cancer cells distinguish themselves from normal cells by their high rate of growing and reproducing new cells. The extreme growth rates required for their rapid reproduction involve massively increased rates of the biochemical reactions supporting the cancerous growth. Each excess reaction produces extra heat and raises the internal cell's temperature and the tissue space immediate to the rapidly growing cells. This heat signature is used as a primary biomarker that enables binding of a nanoviral particle engineered to migrate to at attach at the target site at the site and prevent the cell from continued metabolism. Preferably, the nanoparticle not only binds and is internalized by external membrane receptors on the target cell, but incorporates into the rapidly metabolizing cells additional metabolic blocking agents to stop their growth. When cell growth and proliferation are stopped, the body's natural defenses are able to segregate and eliminate these cells. The massively increased rates of metabolic reactions characteristic of cancer cells also produce greater than normal lactic acid. The resultant decreased pH is useful as a secondary or confirmatory marker for identifying these cancer cells.

Cells are living things. As do all living things cells must conform to laws of chemistry and physics and we have applied laws of nature to describe how living things live by and apply these laws. One accepted law is that a living thing can reproduce or self-replicate, while lifeless entities regardless of their past do not self-replicate. All mammals and other complex organisms, for example homeotherms, like other sexually reproduced organisms begin as one single cell. As we grow and develop this originating cell must grow and produce daughter cells which differentiate, grow and proliferate to produce additional daughter cells continuously throughout our lifetimes. When the growth and proliferation processes become uncontrolled we call this disease phenomenon cancer.

Cancer cells, like all cells, operate as a type of factory reliant on thousands of chemical reactions. These reactions tend to be exothermic and nominally warm the cell and its surrounding tissues. Since cancer cells grow faster than their parent cells, they have a higher heat signature. The accelerated growth rate makes the locally increased temperature common to all cancer cells. The metabolic requirements for rapid proliferation of daughter cells cause normal metabolic paths in cancer cells to shift ATP production in ways that increase H⁺ and reduce ambient pH when the H⁺ and lactate counterion are exported to interstitial space.

The present invention exploits the characteristic local heat signature that occurs in conjunction with increased [H⁺] (decreased local pH) to identify, segregate, isolate and trigger natural elimination of these abnormally hyperproliferating cells.

Growth and proliferation is essential for growth and development and legacy of the species. Like everything, this growth and development procession is not always perfect. One common deleterious anomaly involves uncontrolled cell divisions or hyperproliferation of a lineage of cells in our bodies. We characterize these anomalies as cancers.

Cancer is not a single disease, but cancers are a class of diseases each of which presents a metabolism that has been shifted to support the hyperproliferation that is characteristic of the cancer group. Although different cancers may appear in disparate tissues and cancer cells may migrate from one tissue to another, at their root each cancer cell cohort involves a shift in normal metabolism so that rather than supporting duties to maintain survival of the host organism the cell had differentiated to perform, the cancer cell's metabolism alters pathways, down-regulating several, up-regulating others, to improve the cancer cell's [undesired] hyperproliferative activities.

As an example, each cell division requires another set of nucleic acids to construct a second complete genome. The nucleic acid production pathway must be up-regulated. But the up-regulation of this one pathway will deprive other pathways of their normal resource pools. In view of these considerations, cancer can be thought of as a single disease—counterproductive hyperproliferation with several modes of expression dependent of the initial metabolic status of the cell and the adaptive switches or pressures modifying the initial metabolism to support hyperproliferation. Animal life requires cells to proliferate, but proliferation of the cells must be kept in balance. The present invent addresses the problem of hyperproliferation in two ways: 1) cells adapting their metabolisms along a path towards uncontrolled proliferation are provided stimuli to promote normal metabolic controls of the organism including, but not limited to inducing apoptosis, and 2) inducing the hyperproliferative cell to activate components of the innate immunity system, to facilitate the cells self-destruction and/or to be marked and targeted for attack by the immune system.

While most times our cells, including germ line cells, but also somatic cells, faithfully copy the DNA genetic material to replicate new cells, as part of evolution, our genetic material has been selected to be very, very, slightly unfaithful. In individuals aging is correlated with mutated genomic material. Most mutations do not lead to cancer, but in rare, but still significant cases mutations start a cell down a hyperproliferative pathway that may eventually present as a cancer.

This is relevant to cancer. In cancer a group of cells presents a group of mutations. But cancer itself is not naturally in our genetic material. A specific group of cancer genes is not suddenly switched on. Cancer cells are living things and therefore follow chemical and physical laws and the principles of biology including principles of natural selection.

Cancer itself is a complex disease. A cancer cell is not different in just a single respect from normal desirable cells. Many events are necessary to develop all the changes that make a cell cancerous. Cancer cells have been altered to follow a metabolic program to enhance necessary biosynthesis and support that cell's proliferation. The changes may not be in the best interests of the organism. So concomitant with these metabolic changes must be changes that evade the organism's control of inappropriately behaving cells and that evade the apoptotic cell death protocols carried in each cell's genetic instruction set.

One notable change in rapidly proliferating cells in general, but in cancer cells in particular is a metabolic switch from using the mitochondria for efficient production of adenosine triphosphate (ATP) to favor the less energetically efficient cytoplasmic pathway for ATP production. This alterative pathway produces less ATP per glucose molecule and finishes with lactate, a three carbon molecule, as a chemically energetic metabolite that must be excreted as lactic acid. This requires a protein to transport the lactate ion and hydrogen counter ion across the cell membrane. Lactate/H⁺ is co-transported from inside to outside the cell by one of several monocarboxylate transporter proteins (MCTs).

As mitochondrial ATP production is de-emphasized cytoplasmic pathways, using enzymes evolved for those lactic acid producing pathways, become more active. Generally, expression is accentuated, often at the transcription level, and carried through messenger RNA to ribosomes for synthesis of extra protein copies.

Cancer is a progressive disease of altered metabolisms resulting from mutations in the genetic code of cells developing the disease. As such we have systems to minimize or eliminate mutations. In the nucleus we have systems that detect and correct misreads of the DNA. When mistakes are missed, the cell has systems that recognize imbalances which when severe actually lead that cell to destroy itself to preserve healthy cells of its host organism. However, when these corrective mechanisms fail, the cells will continue to function as programmed by their DNA. When the DNA reprogramming includes hyperproliferation of that cell's lineage and means to evade the organism's controls, tumors can result.

Our immune systems are active against abnormal cells and therethrough are useful for controlling or destroying developing cancers. However, in rare circumstance applying the tenets of survival of the fittest, an especially egregious cancer cell will upregulate systems to minimize the immune attacks against it, from both the intracellular and organism wide levels.

Several papers have evaluated, summarized and reviewed basic anti-immune survival characteristics required for these cells to continue their growth within the organism. For example, as Ribas summarizes:
There is clear evidence that the human immune system can mount cytotoxic immune responses that can eradicate cancers. This indicates that cancers that grow progressively either are not recognized by the immune system or have developed mechanisms to avoid the immune system. Evidence from mouse models of carcinogen-induced cancers led Schreiber and colleagues to postulate the concept of immune-editing, which explains how an otherwise immunogenic cancer can grow progressively.
The concept of adaptive immune resistance is used to describe a process in which tumor antigen-specific T cells attempt to attack the cancer, but the cancer changes in a reactive fashion to protect itself from this immune attack. It was first used by Drew Pardoll to describe how the production of interferons by T cells upon recognition of their cognate antigen results in the reactive expression of the ligand of PD-1 (PD-L1) by cancer cells and the turning off of PD-1-positive T cells.
When tumor antigen-specific T cells recognize their cognate antigen expressed by cancer cells, signaling through the T-cell receptor (TCR) leads to the production of interferons and, at the same time, the expression of activation-induced regulatory receptors, including PD-1. The interferons are aimed at amplifying the immune response and attracting other leukocytes, such as NK cells and macrophages. However, in both mouse models and humans, interferons also lead to the expression of a series of interferon-inducible immune suppressive factors, including PD-L1 and indolamine 2,3 dioxygenase. This is an adaptive process that limits immune and inflammatory responses, and cancer uses it to its advantage.
PD-L1 can be constitutively expressed through a series of currently incompletely analyzed oncogenic pathways, which likely converge in the activation of signal transducers and activators of transcription (STAT) proteins or other interferon receptor downstream effectors, or can be induced in response to both type I and II interferons produced during an active antitumor immune. The interferon inducible expression of PD-L1 seems to be more common than the constitutive expression in most cancer histologies and results in a restricted PD-L1 expression in T cell-rich areas of tumors, in particular at the invasive margin.”

The present invention exploits the metabolic adaptations a hyperproliferating cancer cell requires for its specialized cancerous metabolism to overcome these cellular evasions by identifying, and triggering natural deaths in these abnormally hyperproliferating cells.

Our body's immune system has two functional stages, each with multiple component pathways. The first stage is an “innate response”, a general immune response directed against a range of perceived dangers, e.g., an attack using powerful oxidants. This stage lays in dormant waiting for random foreign organisms. In the second “adaptive response” stage the immune system adapts a response specific to the target, e.g., antibodies that recognize a specific molecular structure.

The innate stage recognizes abnormal molecules presenting in our bodies and initiates a generalized system for eliminating them i) inside a cell with abnormal molecules (such as viral molecules) and ii) recruiting immune killer cells and other immune system cells to the infected cell to perform general anti-microbial tasks around the infected cell.

In one intracellularly initiated innate immune response, pattern recognition receptors (PRRs) inside a cell detect specific viral components such as viral RNA or DNA or viral intermediate products and induce production and secretion of type I interferons (IFNs) (e.g., IFN-α, IFN-β, IFN-ε, IFN-κ and IFN-ω) and other pro-inflammatory cytokines (including, but not limited to: IL-1β, IL-1Ra, IL-4, IL-5, IL-6, IL-7, IL-8, IL-10, IL-13, IL-17, G-CSF, GM-CSF, TNF-α, IP-10, MCP-1, MIP-1α, MIP-1β, RANTES, CCL-2/MCP-1, CCL-4/MIP-1β, CXCL-8/IL-8, CXCL-9/MIG, and CXCL-10/IP-10, keratinocyte-derived chemokine, etc.) in the infected cells and other immune cells to turn on intracellular controls and to signal the body of an attack. [Type II INF-γ, released by immune cells attracted to the infection site, has a secondary effect of potentiating Type I IFN activity and acting as a cytokine for leukocytes.] The innate response is activated within hours of infection and may last for as long as 7 days during a primary influenza infection as the adaptive immune response is being activated.

This innate immune response stage is especially provoked when viral, bacterial or fungal pathogens infect our cells. As one example, influenza virus induces chemokine and cytokine production by infected epithelial cells and monocytes/macrophages. The chemokines attract immune cells, including macrophages, neutrophils and natural killer (NK) cells to the infected location. These cells then release more cytokines, chemokines and other antiviral proteins which provide an additional general killing mechanism as they initiate the adaptive (pathogen specific) immune response.

Type I interferons (IFN-α/β) are major cytokines produced by the innate immune response. They are produced inside an infected cell and by chemokine-recruited immune cells outside the infected cell. These bind internal receptors of the cell that produced IFNs and, when released, plasma membrane receptors on neighbor cells where they induce an enhanced antiviral response. One important early action of IFNs is production of intracellular antiviral proteins that also inhibit protein synthesis in general. This slows all growth including viral reproduction and may initiate an apoptotic event.

These interferons also recruit monocytes/macrophages, T cells and NK cells to the site and also act as signaling molecules to warn nearby cells of the viral presence. This signal induces neighboring cells to increase the numbers of MHC class I molecules upon their surfaces and heighten the immune response. And in the adaptive response they assist maturation of antigen-presenting cells (APCs) and increase expression of major histocompatibility complex (MHC) class I and II molecules on these APCs. These actions, of course, ramp-up antigen presentation for the adaptive immune stage.

The interferon invoked NK cells have a tremendous role in the innate immune response against viral infections. The NK cells are a class of large granular lymphocytes that recognize virus-infected cells in a non-specific manner. Since most infectious viruses down-regulate MHC class I molecules on the surface of infected cells as a survival mechanism to avoid destruction by cytotoxic T Lymphocytes (CTLs), the NK cells counter this by sensing the depletion of MHC class I molecules and then work to induce the infected cell's demise by apoptosis. Apoptosis is a highly orchestrated mechanism whereby a cell disassembles itself into small packages. The apoptotic process includes release of chemokines that attract phagocytic cells such as macrophages to ingest and carry away the disassembled parts.

Apoptosis is advantageous over simple lysis of cells because it limits inflammatory responses. The controlled apoptotic process breaks the cell contents, including contents generated by an infectious virus, into small particles. These are then recycled by the phagocytic cells recruited by cytokines to the infection site. But most non-enveloped viruses, including vaccinia and piconaviruses, initiate host cell lysis, e.g., using a viral encoded viroporin, to break the membrane as their means to release the cell contents including nascent viral particles that will infect neighbor cells.

Nuclear factor-κB (NF-κB) consists of a family of transcription factors that play critical roles in inflammation, immunity, cell proliferation, differentiation, and survival. Bacterial and viral infections (e.g., through recognition of microbial products by receptors such as the Toll-like receptors), inflammatory cytokines, and antigen receptor engagement, all activate NF-κB pathways. General damage to cells, including oxygen stress, γ-radiation and ultraviolet light also induce the pathway.

The activity of NF-kB and its pathways is primarily regulated by interaction with inhibitory IkB proteins. In the quiescent state, NF-κB dimers (e.g., NF-κB2 (p52/p100), NF-κB1 (p50/p105), c-Rel, RelA (p65), RelB in mammals) are sequestered in the cytoplasm by specific inhibitors—the IκBs. The “classical” pathway is activated by a collection of diverse stimuli including, but not limited to: cellular stress, cytokines, free radicals, UV radiation, oxidized LDL, bacterial/viral infection, etc. When IκB kinase (IKK) is activated, IKK phosphorylates NF-κB-bound IκBs thereby marking them for rapid ubiquitin-dependent degradation when it forms a binding site for the SCF^{βTrCP} ubiquitin ligase complex.

The no-longer inhibited NF-κB dimers can then enter the nucleus to coordinate the transcriptional activation of several hundred genes including cell death/apoptotic genes for Bcl-XL, Bcl-2, and X-linked inhibitor of apoptosis protein (XIAP). This NF-κB signaling pathway, is only one of three pathways that activate NF-κB transcription factors.

The second pathway, named the “alternative” pathway, specifically activates p52:RelB heterodimers. Unlike the classical pathway, which is absolutely dependent on IKKγ and to a large extent on the IKKβ catalytic subunit, the alternative pathway requires activation of IKKα homodimers whose preferred substrate is the precursor for p52—the p100/NF-κB2 protein. The NF-κB2 precursor binds RelB through its N-terminal Rel homology domain and this complex is retained in the cytoplasm through its C-terminal IκB-like domain.

The third pathway leading to NF-κB activation is IKK-independent and instead is based on activation of casein kinase 2 (CK2), which induces IκBα degradation through phosphorylation of C-terminal sites. Although this pathway is only a minor contributor to NF-κB activation, it may be of importance especially in skin carcinogenesis because it is activated by UV radiation.

The innate immune system has both intracellular and extracellular components. The lethality of the 1918 pandemic influenza virus has been associated with insufficient innate intracellular response and extreme levels of virus replication resulting in severe lung inflammation and prolific infiltration into the lungs of neutrophils and alveolar macrophages. This severe outcome seems to be the result of an especially inefficient intracellular innate immune response to this subtype of influenza. The limited efficacy of the innate immune response against the 1918 virus probable resulted from the adaptation of the virus NS1 gene to suppress the IFN-α/β system thereby permitting the virus to reproduce without immune restraints.

Early in infection, viral NS1 binds to the PI3K subunit p85 and activates the kinase which drives phosphorylation and activation of Akt/PKB by the phosphorylated pyruvate dehydrogenase kinase. When activated the Akt down regulates pro-apoptotic factors such as caspase 3, caspase 9, Bcl-2 associated death promoter (BAD), and GSK-3 to suppress apoptosis while the virus proliferates is genome. At later stages of infection, NF-κB activation induces expression of pro-apoptotic factors, e.g., Fas, FasL, and TRAIL and subsequent caspace induction.

The influenza B induced path to apoptosis through caspace 8 early in the infection process reveals a minimized ability to prevent host cell death at this stage of infection.

All self-propagating organisms have developed mechanisms to protect themselves from invasion/takeover by exogenous microorganisms. Multiple cytokines and chemokines are produced by several kinds of host cells in viral infections, but type I IFNs are the apparent principal cytokines in the mammalian antiviral response. Type I IFNs include multiple IFN-α isoforms, a single IFN-β, and other members, such as IFN-ε, -κ, -ω, etc. In contrast to type II IFN (IFN-γ), exclusively produced by T cells and NK cells, type I IFNs can be produced by all nucleated cells when challenged with viral infection. Type III IFNs, comprising IFN-λ1, λ2 and λ3, have also recently been identified as receptors recognizing molecular patterns peculiar to microorganisms, such as viral genome nucleic acids.

Nucleic acids, e.g., DNAs and RNAs are essential for survival of all known living organisms. The nucleic acid defines the organism for what it is and what it can do. Therefore discrimination between self and non-self DNAs/RNAs is an essential self defense, especially in the virus infection process which involves the viral particle using the host cell machinery (including its nucleic acids and products thereof) to promulgate the viral genome. This self/non-self discrimination relies on pattern recognition receptors (PRRs).

PRRs are proteins expressed by cells of the organism's innate immune system, e.g., DCs (DCs), macrophages, monocytes, neutrophils and epithelial cells. PRRs identify and bind two patterns of molecules: pathogen-associated molecular patterns (PAMPs), which are associated with microbial pathogens, and damage-associated molecular patterns (DAMPs), which are associated with components of host's cells that are released during cell damage or death. PRRs also participating in initiation of the antigen-specific adaptive immune response and release of inflammatory cytokines. These cytokines include, but are not limited to: Toll-like receptors (TLRs), retinoic acid-inducible gene I (RIG-I)-like receptors (RLRs), and nucleotide-binding oligomerization domain (NOD)-like receptors (NLRs). Here, we review the current understanding of innate immune recognition of viruses and discrimination between self and viral nucleic acids, and provide some recent advances in coordination between innate immune signaling and adaptive immune activation.

Virtually all nucleated cell types in the body have the machinery to present as “non-professional antigen presenting cells” (NPAPCs). These are distinguished from “professional antigen presenting cells” (PAPCs) which couple an MHC class I molecule to β-2 microglobulin to display endogenous peptides on the cell membrane. PAPCs when attracted to an infection site are efficient internalizers of antigens, e.g., by phagocytosis (macrophages and dendritic cells) or by receptor-mediated endocytosis (B cells). They chop the antigen into peptide fragments and then display those peptide fragments on the cell surface. PAPCs generally express class II MHC while NPAPCs generally present antigens using class I MHCs.

The peptides presented by the ordinary cells that became NPAPCs as a result of infection originate from within the infected cell itself. MHC class I molecules present antigen using class I MHCs as a marker for cytotoxic T cells then are able to interact with the antigen presented using an MHC class I molecule. Antigenic peptides that bind to MHC class I molecules arise from viruses that commandeer the biosynthetic machinery of the host cell to produce the foreign antigens. These foreign antigen/viral proteins are degraded by the host cell's proteasomes into small peptide fragments. MHC class I molecules, produced in response to infection, then associate with Transporters Associated with Antigen Processing-1 or -2 (TAP-1 or TAP-2) and transported into the endoplasmic reticulum (ER) where they bind to the MHC molecule, for transport through the Golgi apparatus to the plasma membrane for interaction with T cells. Cytotoxic T cells regularly migrate through the body ready to bind cells presenting MHC class I bound foreign antigens. The cytotoxic T cells then interact with the presenting cell to initiate or support apoptosis of the infected cell. Viruses selected or engineered to have more robust antigen for more efficient or more rapid T cell recruitment towards presenting cell apoptosis may be included in a preferred embodiment.

Alan Epstein and Leslie Khawli in U.S. Pat. No. 9,522,958 propose using our natural innate immunity by attaching a nucleotide to a “cancer targeting molecule”, defined as a molecule that has the ability to localize to cancer cells in an individual. They discuss the background biology with reference to innate immunity and then after providing several specific nucleic acid stimulants as examples characterize CpG-immunostimulatory oligonucleotides as being well known in the art:
Immunotherapy (sometimes called biological therapy, biotherapy, or biological response modifier therapy), which uses the body's immune system, either directly or indirectly, to shrink or eradicate cancer has been studied for many years as an adjunct to conventional cancer therapy. It is believed that the human immune system is an untapped resource for cancer therapy and that effective treatment can be developed once the components of the immune system are properly harnessed. As key immunoregulatory molecules and signals of immunity are identified and prepared as therapeutic reagents, the clinical effectiveness of such reagents can be tested using well-known cancer models. Immunotherapeutic strategies include administration of vaccines, activated cells, antibodies, cytokines, chemokines, as well as small molecular inhibitors, anti-sense oligonucleotides, and gene therapy (Mocellin, et al., Cancer Immunol. & Immunother. (2002) 51: 583-595; Dy, et al., J. Clin. Oncol. (2002) 20: 2881-2894, 2002).
The growth and metastasis of tumors depends to a large extent on their capacity to evade host immune surveillance and overcome host defenses. Most tumors express antigens that can be recognized to a variable extent by the host immune system, but in many cases, the immune response is inadequate. Failure to elicit a strong activation of effector T-cells may result from the weak immunogenicity of tumor antigens or inappropriate or absent expression of co-stimulatory molecules by tumor cells. For most T-cells, proliferation and IL-2 production require a co-stimulatory signal during TCR engagement, otherwise, T-cells may enter a functionally unresponsive state, referred to as clonal anergy.
As part of the immune system, innate immunity provides an early first line defense to pathogenic organisms which is followed by antibody and cellular T cell responses characteristic of the adaptive immune system. Innate immunity is highly robust and utilizes specific cells such as macrophages, neutrophils/PMNs, DCs, and NK cells which are effective in destroying and removing diseased tissues and cells (Cooper et al., BioEssays (2002) 24:319-333).
These microbial stimulators of innate immune responses include lipopolysaccharides and teichoic acids shared by all gram-negative and gram-positive bacteria, respectively, unmethylated CpG motifs characterized by bacterial but not mammalian DNA, double-stranded RNA as a structural signature of RNA viruses, and mannans which are conserved elements of yeast cell walls. None of these structures are encoded by host organisms and all are shared by large groups of pathogens due to their importance in structure and/or propagation of the infecting organism. Mammals have developed a set of receptors which recognize these microbial components. Unlike T- and B-cell receptors of the adaptive immune system, however, these innate system receptors are germline encoded (since they have arisen evolutionarily over time due to selection by pathogens at the population level) and are strategically expressed on cells that are the first to encounter pathogens during infection (Ozinsky, et al., PNAS (2000) 97:13766-13771).
CpG Oligodeoxynucleotides (ODNs) are synthetic oligonucleotides that are comprised of unmethylated CG dinucleotides, arranged in a specific sequence and framework known as CpG motifs (Tokunaga, et al., JNCI (1984) 72:955-962; Messina, et al, J. Immunol. (1991) 147:1759-1764; Krieg, et al, Nature (1995) 374:546-549). CpG motifs trigger the production of T-helper 1 and pro-inflammatory cytokines and stimulate the activation of professional antigen-presenting cells (APCs) including macrophages and DCs (Klinman et al. PNAS (1996) 93:2879-2883). Unmethylated CpG ODNs behave as immune adjuvants which accelerate and enhance antigen-specific antibody responses and are now thought to play a large role in the effectiveness of Freund's Adjuvant and BCG (Krieg, Nature Med. (2003) 9:831-835). Recently, it was discovered that CpG ODNs interact with Toll-like receptor (TLR) 9 to trigger the maturation and functional activation of professional antigen presenting cells, B-cells, and natural killer cells (Hemmi, et al. Nature (2000) 208:740-745; Tauszig, et al, PNAS (2000) 97:10520-10525; Lawton and Ghosh Current Opin. Chem. Biol. (2003) 7:446-451). CpG ODNs are quickly internalized by immune cells, through a speculated pathway involving phophatidylinositol 3-kinases (PI3Ks), and interact with TLR9 present in endocytic vesicles (Latz, et al. Nature Immunol. (2004) 5:190-198). The resultant immune response is characterized by the production of polyreactive IgM antibodies, cytokines, and chemokines which induce T-helper 1 immunity (Lipford, et al., Eur. J. Immunol. (1997) 27:2340-2344; Weiner, J. Leukocyte Biol. (2000) 68:445-463; Stacey, et al., Curr. Topics Microbiol. Immunol. (2000) 247:41-58; Jacob, et al., J. Immunol. (1998) 161:3042-3049). The TLR9 receptor recognizes CpG ODNs with a strict bias for the chemical and conformational nature of the unmethylated CpG ODN since conjugation of an oligonucleotide and a CpG DNA at the 5′-end has been shown to reduce significantly the immunostimulatory activity of the CpG DNA. On the other hand, conjugation of an oligonucleotide and a CpG ODN at the 3′-end does not perturb or may even enhance the immunostimulatory activity of the CpG DNA (Kandimilla, et al., Bioconjug. Chem. (2002) 13:966-974).
An immunostimulatory sequence motif which contains at least one unmethylated CG dinucleotide refers to the portion of an oligonucleotide that includes the unmethylated CG dinucleotide and several nucleotides on each side of the CpG that are critical for the immunostimulatory activity. For example, the immunostimulatory motif containing the CG dinucleotide is shown bolded and italicized with the CpG bolded and underlined in the following sequence: 5′-TCGTCGTTT-3′.
Oligonucleotides which comprise an immunostimulatory sequence motif that contains at least one unmethylated CG dinucleotide have been referred to the in art as “oligodeoxynucleotide containing unmethylated CpG motifs,” or “CpG oligodeoxynucleotides (“CpG ODNs”). The phrase “oliognucleotide comprising an immunostimulatory sequence motif which contains at least one unmethylated CG dinucleotide” may be referred to herein as a “CpG immunostimulatory oligonucleotide.”
Cells stimulated by CpG immunostimulatory oligonucleotide secrete cytokines and chemokines (IL-1, IL-6, IL-18 and TNF) including Th1-biased cyokines (interferon-γ, IFN-γ, and IL-12) to create a pro-inflammatory immune response (Klinman, Nature Rev. Immunol. (2004) 4:249-258). Also stimulated are professional antigen-presenting cells (APCs) which include macrophages and DCs (Krieg, et al., Nature (1995) 374:546-549; Klinman, et al. PNAS (1996) 93:2879-2883).
The CpG ODN contain one or more unmethylated CG dinucleotides arranged within a specific sequence (Tokunaga, et al., JNCI (1984) 72:955-962; Messina, et al, J. Immunol. (1991) 147:1759-1764; Krieg, et al, Nature (1995) 374:546-549). The optimal CpG flanking region in mice consists of two 5′ purines and two 3′ pyrimidines, whereas the optimal motif in humans and certain other species is TCGTT and/or TCGTA (Klinman, Nature Rev. Immunol. (2004) 4:249-258). The CpG immunostimulatory oligonucleotide is generally from 6 to 100 nucleotides in length, more preferably between about 15 to 25 nucleotides in length. As described by Sen et al., (Cell Immunol. 2004 November-December; 232(1-2):64-74), portions of an oligonucleotide that has immunostimulatory motifs containing an unmethylated CpG can be replaced with RNA. For example, the RNA can be used in the oligonucleotide to flank the critical immunostimulatory motif.
Oligonucleotides comprising an immunostimulatory sequence motif which contains at least one unmethylated CG dinucleotide and have in vivo immunostimulatory activity may be used to prepare invention conjugates. In some embodiments, the oligonucleotide may be chemically modified to enable linkage to the cancer targeting molecule. Modification may involve adding a thiol group to the 3′ terminal nucleotide using a non-nucleoside linker (3′-thiol-modifier C3) (Zukermann et al., Nucleic Acids Res, 15: 5305-5321, 1987) to facilitate covalent linkage with linker modified antibody. The following CpG immunostimulatory oligonucleotides are exemplary (CpG motifs identified by bolded text with underlining).

### INCORPORATED BY REFERENCE

CpG immunostimulatory oligonucleotides having applications for animal use include class A, B or C type CpG ODNs which are well known and may linked to a cancer targeting molecule as described herein.

One perennial challenge and activator of our innate and adaptive immune systems is the “annual” human flu virus. “Annual” because the virus rapidly changes in adaptive response to evade our adaptive immunities developed in response to previous virus versions. Influenza A and B viruses, the most common for infecting humans, comprise a single-stranded, negative-sense RNA genome with eight RNAs that encode 10-11 proteins. Similar strains have adapted to infect birds and other domesticated animals such as mammals, e.g., dogs, cats, etc. The virions are enveloped with two surface glycoproteins, HA and NA. These attach and release the virus from host cells, respectively. The adaptive phase emphasizes antibodies against these glycoproteins. The third transmembrane viral protein, M2, is a miniscule component of the envelope, typically with a score or fewer protein molecules in the entire envelope.

Each of the 8 gene segments in the virion is associated with three polymerase proteins (PB2, PB1, and PA) and a nucleoprotein (NP). The polymerase complex mediates the nuclear transport of the viral ribonucleoproteins (vRNPs) to facilitate viral transcription.

Influenza A viruses encode for the 11 viral genes: hemagglutinin (HA), neuraminidase (NA), matrix 1 (M1), matrix 2 (M2), nucleoprotein (NP), non-structural protein 1 (NS1), non-structural protein 2 (NS2; aka nuclear export protein, NEP), polymerase acidic protein (PA), polymerase basic protein 1 (PB1), polymerase basic protein 2 (PB2) and polymerase basic protein 1-F2 (PB1-F2).

The cell ligand, HA, appears in trimers on the viral membrane which bind to sialic acid (SA) on the surface of the host cell's membrane. Two major linkages are found between sialic acids and the carbohydrates they are bound to in glycoproteins: α(2,3) (birds, horses, pigs) and α(2,6) (humans, pigs).

The endosomal process of viral entry favors fusion of the viral and endosomal membranes at low pHs. Low pH also induces a conformational change in HA0 exposing the HA2 fusion peptide. When the fusion peptide inserts itself into the endosomal membrane it brings the viral and endosomal membranes into contact with each other. The acidic environment also opens up the M2 ion channel that acts as a proton-selective ion channel acidifying the viral core. The low pH releases the vRNP from M1 to enter the host cell's cytoplasm.

Nuclear access is facilitated by nuclear localization signals (NLSs) on the NP, PA, PB1, and PB2 components of the vRNP. The NLSs are transported into the nucleus by the cell's intracellular transport machinery. In the nucleus the viral RNA-dependent RNA polymerase (RdRp) initiates complementary RNA synthesis internally on viral RNA. Viral (positive) RNA leaves the nucleus and is transported to the ribosomes to manufacture the new viral proteins —nucleoproteins which return to the nucleus for packaging with negative strand viral RNA remaining there and the viral surface proteins that are transported to the cell membrane post manufacture. These combine within the cell membrane and are released by budding off the membrane is a steady progression. This contrasts with the bulk release typical of non-enveloped viruses (like vaccinia) which distribute by lysing their host cell. Since viral assembly takes place on the cell membrane in preparation for budding, viral proteins, especially HA and NA, are exposed on the host cell surface. Recognition of these foreign proteins in soon to be budded rafts is one means through which the infected cells can be targeted by the host organism immune system. These proteins are thus preferred targets for selection/engineering for improved recognition and destruction of the targeted cells.

Alternatively, an infected cell may be or become uncooperative at viral replication often initiating its own apoptotic cell death as an innate immune process. The innate immune response of the cell includes the pattern recognition receptors (PRRs), including, but not limited to: TLR3, TLR7, IRF7, MDA5, RIGI, etc., that, when they sense incoming viruses, activate transcription of interferon (IFN) genes, including, but not limited to: IFNB1, IL28A, IL29, IL28B, IFNW1, IFNA7, IFNA14, IFNA10, IFNA13, IFNA16, IFNA8, IFNA1, IFNG, IFNA2, IFNA21, etc. IFNs cause the cell to express its interferon stimulated genes (IFGs) to produce many types of anti-pathogenic proteins. For example: IFITM1 and SAMD9 which interfere with fusion between viral and endosome membranes; HERC5, HERC6, USP18, ISG15, TRIM22, and ISG20 which tag viral proteins for degradation and, thereby, mediate viral RNA (vRNA) uncoating; IFIT1, IFIT2, OASL, IRF7, DDX60, DDX58/RIG-I, IFIH1/MDA5, and EIF2AK2/PKR which recognize vRNA, and OAS1, OAS2, and OAS3 which then degrade the vRNA; ZBP1, PARP1, PARP9, PARP14, and PRIC285 which inhibit transcription and translation of vRNA; lipid raft-disturbing factor RSAD2 which prevents assembly of vRNPs in the host membrane; cholesterol-depleting factor IFITM3 which inactivates budding viruses; apoptosis regulators IF127 and XAF1; IDO, COX2, and CH25H that produce neuro- and immuno-modulators; multiple cytokines and chemokines for activation and recruitment of immune cells to the site of infection; etc.

To improve its survival chances the virus employs NS1 to block the transcription of innate antiviral genes by its direct binding with the cellular DNA and interaction with vRNA and its replication intermediates to prevent its recognition by cellular PRRs and the cell's defensive RNAses. A preferred engineered version of the virus comprises a weakened NS1 to elicit a more robust innate immune response, including IFN-1 production, in the engineered infection.

A secondary innate response system of the cell comprises activating its apoptosis process whereby the cell turns off most of its entire metabolism preventing the cell from creating new viruses that may infect other cells and organisms. The cell, by ceasing most metabolism, essentially kills itself and along with it the machinery to propagate new viral particles.

When the virus escapes the IFN responses e.g., through activation of its NS1, PRRs recognize accumulating vRNA and activate apoptotic machinery that directs the fate of IAV-infected cells to self-destruct. The anti-apoptotic (Bcl-2, Bcl-xL, and Bcl-w) and pro-apoptotic (Bax, Bak, Bad, Bim, Bid, Puma, and Noxa) Bcl-2 proteins associate or dissociate, respectively, to start a cascade of reactions that result in mitochondria membrane permeabilization (MoMP) and release of cytochrome c into the cytoplasm, with subsequent apoptosome activation, ATP degradation, and cell death. Presence of other apoptosis stimulants, e.g., chemical and physical stressors, nitric oxide, UV light, oxygen stress, temperature, may be facilitated to synergize the effects of the engineered virus. Several biologics, small molecule drugs and drug prototypes including, but not limited to: YCKVILTHRCY, GRVCLTLCSRLT, cannabidiol, kaempferol, URB937, Costunolide, TW-37, Epibrassinolide, 2-arachidonoylglycerol, 15-acetoxy Scirpenol, NSC 687852 (b-AP15), Cycloheximide, Bendamustine HCl, CFM 4, 7BIO, MPI-0441138, Citrinin, Destruxin B, (±)-Jasmonic Acid methyl ester, Psoralidin, JWH-015, ML-291, F16, Mitomycin C, Betulinic acid, BAM7, Kaempferol, Gambogic Acid, Apicidin, 2-Methoxyestradiol (2-MeOE2), Kaempferol, dexamethasone, 3,3′-Diindolylmethane, Brassinolide, Capsaicin, Triciribine Curcumin, Matrine, R1530, SMIP004, Trabectedin, 2,3,7, 8-tetrachlorodibenzo-p-dioxin, PM00104, Meisoindigo, 2,3-DCPE hydrochloride, Actinomycin D, Raltegravir potassium salt, C 75, Atractyloside Dipotassium Salt, CHM 1, Deguelin, Oncrasin 1, Streptozocin, Piperlongumine, FAAH inhibitors, perforin, Gambogic Acid, Linoleic Acid, PKC-412, Z3902, V9389, T7329, T2577, SRP5180, SRP5168, SRP5166, SRP5164, SRP4928, SRP3199, SRP3047, SRP3046, SML1908, SML1903, SML1843, SML1827, SML1823, SML1793, SML1765, SML1758, SML1745, SML1710, SML1707, SML1660, SML1637, SML1635, SML1601, SML1576, SML1533, SML1493, SML1492, SML1490, SML1464, SML1456, SML1372, SML1306, SML1302, SML1269, SML1263, SML1187, SML1156, SML1131, SML1016, SML1013, SML0991, SML0978, SML0963, SML0954, SML0953, SML0932, SML0907, SML0892, SML0821, SML0641, SML0623, SML0610, SML0580, SML0552, SML0521, SML0507, SML0433, SML0417, SML0404, SML0367, SML0363, SML0256, SML0188, SML0140, SML0096, SML040, SML031, SMB00431, SMB00418, SMB00388, S7451, S7448, R9156, R5030, R3530, PZ0115, P1499, P0103, P0069, N9162, N6287, M7888, K4394, 17160, 15159, H8787, H4663, G8171, G7923, G7548, F9428, E9661, E7781, E5411, E5286, E5161, E4660, D7446, D5817, C9369, C7744, C5865, C5492, C4992, C1244, BM0018, B8809, B5936, B5437, B3061, B0261, A8476, A4233, A3105, etc., are easily synthesized, purified from available products or available from suppliers to augment the apoptotic pathways. The virus may preferably be engineered to inhibit or eliminate its anti-apoptotic pathway effects and/or to facilitate/inhibit pro/anti-apoptotic protein paths in the infected cell. Apoptosis augmenters may be used cooperatively for additive or synergistic effect in killing the hyperproliferative cells.

Viral NP, its most abundant protein, contributes to influenza infection induced cell death; heterologous expression of NP alone can induce apoptosis in culture. Different versions of influenza proteins PB1-F2, NS1, M1, M2 and NA present different modulatory effects on cells' apoptotic processes. Using virus selected or engineered to minimize anti-apoptotic proclivity is one tool for facilitating death in the targeted cells. Accelerating the cells' expression of its more pro-apoptotic proteins is another selection tool.

A virus engineered, e.g., by targeted mutagenesis or serial passaging, with elevated presence of CpG-immunostimulatory oligonucleotides provides a more robust intracellular response.

PB1-F2 protein of the virus translocates to the mitochondrial inner membrane where it facilitates apoptosis within host cells. This is believed to be an adaptive process of the viral life cycle to kill infected cells after virion is budded but to prevent cytokine production and release and minimize adaptive immune activities.

The awesomely deadly 1918 H1N1 pandemic—responsible for 50 to 100 million human deaths—may have derived from a virus of avian origin that after accumulating multiple adaptive mutations became competent to infect several and then to efficiently spread between humans. The 1957 H2N2 pandemic arose when a circulating human influenza virus acquired the H2, N2, and PB1 genes from an avian influenza virus. The 1968 H3N2 pandemic occurred after a circulating human influenza virus acquired the H3 and PB1 genes from an avian influenza virus.

Previous pandemic viruses crossed species barriers after acquiring mutations that changed the binding preference of the HA from avian-like, α-2,3 Sialic Acid (SA), to human-like, α-2,6 SA. Some recently identified subtypes of avian influenza viruses have caused limited human infections, but none have acquired the capacity for efficient and sustained transmission among humans, a key property of a pandemic virus.

As seen in the above relating to humans, in nature, viruses, such as flu viruses, are not static. They constantly morph and continue to improve capacities to propagate more viral entities and to afflict other species. In this endeavor the viruses modulate their methods for controlling the resultant host cell's virus supporting metabolisms.

In addition to these natural viral changes, man has directed and controlled viral changes affecting, for example, host cell recognized by virus, and other means of replication and dispersal. For example, Sander Herfst et al, in their paper: “Airborne Transmission of Influenza A/H5N1 Virus Between Ferrets” published in Science 2012 describe some available methodologies used for directed viral adaptation. In essence two main concepts guide the new viral creations: a) selecting conditions for the virus to self-select according to survival of fittest principles and b) introducing genetic material or mutations into the viral genome. They used both targeted mutagenesis and serial passaging to select viral substrains advantageously growing in the passage target cell:
Using a combination of targeted mutagenesis followed by serial virus passage in ferrets, we investigated whether A/H5N1 virus can acquire mutations that would increase the risk of mammalian transmission. We have previously shown that several amino acid substitutions in the RBS of the HA surface glycoprotein of A/Indonesia/5/2005 change the binding preference from the avian α-2,3-linked SA receptors to the human α-2,6-linked SA receptors . . . . Passaging of influenza viruses in ferrets should result in the natural selection of heterogeneous mixtures of viruses in each animal with a variety of mutations: so-called viral quasi-species.

In a similar influenza engineering exercise, Ron Fouchier et al reported producing an engineered H5N1 virus with massively increased ability to spread amongst humans.

The human capacity to engineer viruses, including influenza viruses, has been demonstrated in these and multiple additional laboratory exercises. The result(s) of manipulations in the viral genomes therefore offer promising information and great potential for using selected and/or engineered viruses for benefit of man.

For example, the influenza virus may have its RNA mutated using site specific mutagenesis of one or several RNA bases or by substituting in whole segments of RNS, including an entire molecule. Selective growth and serial passaging may be used to duplicate one or in some cases a couple entire influenza genes. Such duplicate may comprise a perfectly identical pair or may comprise genes capable of directing expression of different proteins.

The selected/engineered genes may result in a varied induction within target cells, e.g., with different timing of expressed cellular response proteins, amount of protein expressed, species of protein expressed, etc. The innate immunity, including, but not limited to: interferons, cytokines, lymphokines, peptidylglycan recognition proteins, pattern recognition factors, interleukins, TLRs, etc., of the cell is thereby controllable by infection with one or more selected/engineered virus. In several instances the description in this application will use the slashed version “selected/engineered” as a reminder of the equivalency of result regardless of the term conveniently used. The reader will understand that selection may be considered one version of engineering or a part of the engineering process and thus the terms will often be considered equivalent when one or other appears without its slashed partner.

Type I interferons are essential to innate resistance to influenza virus infection and the subsequent induction of adaptive immunity effector responses. Viral RNA products generated during infection are recognized by Toll-like receptors (TLRs) and retinoic acid-inducible gene I (RIG-I) like receptors (RLR) to initiate the interferon response.

Airway epithelial cells recognize the double-strand RNA and/or 5-O-triphosphate ssRNA via RIG-I, a cytosolic RNA helicase, resulting in production of type-I IFN through an adapter protein IPS-1.

Host cells recognize the invasion/internalization of viruses and respond with strong antiviral activities. Viruses initially activate the innate immune system, which recognizes viral components through PRRs. On the other hand, acquired immunity plays a major role in the responses to re-infection with viruses. Host PRRs detect viral components, such as genomic DNA, single-stranded (ss) RNA, double-stranded (ds) RNA, RNA with 5′-triphosphate ends and viral proteins.

Three classes of PRRs have been shown to be involved in the recognition of virus-specific components in innate immune cells, namely Toll-like receptors (TLRs), retinoic acid-inducible gene I (RIG-I)-like receptors (RLRs) and NOD-like receptors (NLRs). TLRs and RLRs are leaders for production of type I interferons (IFNs) and various cytokines, whereas NLRs are known to regulate interleukin-1β (IL-1β) maturation through activation of caspase-1.

Detection of viral components by RLRs and TLRs in immune cells activates intracellular signaling cascades. This elicits secretion of type I IFNs, pro-inflammatory cytokines and chemokines, and increased expression of co-stimulatory molecules such as CD40, CD80 and CD86. Type I IFNs activate intracellular signaling pathways via a type I IFN receptor, and regulate the expression of a set of genes. The IFN-inducible genes, such as protein kinase R and 2′5′-oligoadenylate synthase, are involved in eliminating viral components from infected cells and inducing apoptosis of infected cells. Type I IFNs are produced not only by innate immune cells, including DCs (DCs) and macrophages, but also by non-immune cells, such as fibroblasts.

Proinflammatory cytokines and chemokines are also critical for eliminating virus infection by provoking inflammation and recruiting innate and acquired immune cells. Co-stimulatory molecules are essential for the activation of T cells.

In addition to the RLRs, TLRs are important for recognizing virus infection. TLRs comprise a) LRRs, a transmembrane domain and b) a cytoplasmic domain designated the Toll/IL-1 receptor (IL-1R) homology (TIR) domain. TLRs are transmembrane proteins suitable for detecting viral components outside of cells or in cytoplasmic vacuoles after phagocytosis or endocytosis. Among the TLRs present in mammals, TLR2, TLR3, TLR4, TLR7 and TLR9 appear most involved in recognition of viral components. TLR2 and TLR4, on plasma membrane, recognize viral envelope proteins on the cell surface; while TLR2 and TLR4 recognize bacterial components, lipoproteins and lipopolysaccharide. TLR3, TLR7 and TLR9 are localized on cytoplasmic vesicles, such as endosomes and the endoplasmic reticulum (ER), and recognize microbial nucleotides internally. TLR3 recognizes dsRNA, while TLR7 and TLR9 recognize ssRNA and DNA with CpG motifs, respectively.

The TLRs except TLR3 activate a common signaling pathway leading to the production of proinflammatory cytokines via MyD88, a protein comprised of a N-terminal death domain (DD) and a C-terminal TIR domain. Upon ligand stimulation, MyD88 interacts with IL-1R-associated kinase (IRAK)-4. Humans have 4 IRAK family members, IRAK-1, IRAK-2, IRAK-M and IRAK-4. The IRAKs are characterized by an N-terminal DD and a C-terminal serine/threonine kinase domain. IRAK-4 is an upstream kinase that phosphorylates IRAK-1 and IRAK-2. IRAK-1 rapidly interacts with IRAK-4 and is phosphorylated after TLR activation, and then IRAK-1 undergoes degradation by the ubiquitin-proteasome pathway. In contrast, IRAK-2 interacts with IRAK-4 later than IRAK-1, and stayed phosphorylated for a long time. IRAK-2−/− macrophages failed to sustain cytokine gene expression in response to TLR stimulation, and cells lacking both IRAK-1 and IRAK-2 show abrogated TLR-mediated cytokine production as well as severe impairment in NF-κB activation. These results indicate that IRAK-1 and IRAK-2 are sequentially activated by IRAK-4, and are essential for the TLR signaling. On the other hand, IRAK-M is reported to be a negative regulator of the TLR signaling. Parallel pathways are present in most complex organisms, especially homeothermic organisms kept as domesticated animals.

Downstream of IRAKs, TRAF6 is activated and catalyzes the formation of a K63-linked polyubiquitin chain on TRAF6 and on IKK-γ/NF-κB essential modulator (NEMO), together with an ubiquitination E2 enzyme complex consisting of UBC13 and UEV1A (69). TRAF6 also activates TGF-β-activated kinase 1 (TAK1), which phosphorylates IKK-β and MAP kinase kinase 6, which modulates the activation of NF-κB and MAP kinases that results in induction of genes involved in inflammatory responses. Deletion of TAK1 and UBC13 in mice revealed that these molecules play a critical role in TLR-mediated cytokine production, in addition to their role in embryonic development (70, 71). TAK1 is essential for both NF-κB and MAP kinases, whereas UBC13 was dispensable for NF-κB activation.

Influenza virus has also been characterized in its activation of host adaptive immune responses. Induction of type I IFNs, e.g., in response to intranasal influenza A virus infection was found to be abrogated in the absence of both MyD88 and IPS-1.

Antiviral immune responses in vivo are mediated not only by DCs, macrophages, T cells and B cells, but also by many other cell types, such as NK cells and NK T cells.

Glick and Franchi provide a description of a cellular component to this innate system in the recent published patent application US 20170056448:

Innate Immune Cells
Innate immune cells are mammalian cells that do not recognize pathogenic material (e.g., cancer cells, bacteria, viruses, and yeast) by expressing an antibody or a TCR on its cell surface. Innate immune cells expresses receptors (e.g., receptors on its cell surface) or proteins that bind to the Fc region of other antibodies that are bound to a pathogen and/or receptors that bind to PAMPs that are associated with pathogens and/or DAMPs that are associated with damaged or transformed cells. Non-limiting examples of DAMPs include nuclear or cytosolic proteins (e.g., HMGB1 protein or S100 protein), DNA or RNA, purine metabolites (e.g., ATP, adenosine, or uric acid), and glycans or glycoconjugates (e.g., hyaluronan fragments). Non-limiting examples of PAMPs include bacterial lipopolysaccharide, flagellin, lipoteichoic acid, peptidoglycan, double-stranded RNA, and unmethylated CpG motifs. Additional examples of PAMPs and DAMPs are known in the art.
Non-limiting examples of innate immune cells include mast cells, macrophages, neutrophils, DCs, basophils, eosinophils, and natural killer cells. Additional examples of innate immune cells are known in the art.

Cancer cells arise from diverse tissues and from many, many cell types, but at the root of any cancer is that cell's increased rate of making new cells, that is: hyperproliferation. Every time a cell proliferates it splits to create two cells each of which requires its own membrane, cytoskeleton, nucleus, mitochondria and other organelles. This duplication requires the cell to accelerate synthetic pathways and several additional pathways that support accelerated synthesis. The resulting two cells will require a doubling of DNA for duplicated nuclei, additional membrane lipids and proteins to cover the increased surface/volume ratio, extra endoplasmic reticulum, golgi, mitochondria, lysosomes, etc. to be split between two cells during mitosis. Mitosis itself is a resource hungry process requiring a slew of catabolic and anabolic events. In essence a metabolic rush is necessary to provide an additional set of all cellular components and the temporary resources and energy necessary to divide the cell into two. This accentuated metabolism can be employed to guide intercourse between an interested party and the cancerous or precancerous metabolically modulated cell(s).

This situation is aptly described in US patent application 20040253323 16 Dec. 2004 by Brian Giles:

Background Information and Discussion of Related Art
Cancer cells are different from normal healthy cells in several respects. One way in which virtually all cancer cells differ from normal healthy cells is that cancer cells derive a major proportion of their energy from glycolysis. Normal healthy cells utilize an oxidative metabolism in which only a small proportion of energy is derived from glycolysis. Cancerous neoplasm's require an alteration of energy production with transition from non-invasive premalignant to invasive malignant morphology, ranging from large benign tumors to necrotic cancers, including the acquisition of angiogenesis, increased glucose utilization (with increased lactic acid production) and typical tumor morphologies.
The increased lactic acid production of tumors causes the micro environment outside the tumor edge to become more acidic, leading to reduced pH. This decreased pH kills the normal tissue cells, which surround the tumor and which require a pH of 7.31 or higher to stay healthy and viable. As a consequence, the tumor is surrounded by necroticised normal cells. If insufficient alkalinizing agents are available to the healthy tissue cells surrounding the tumor, this promotes the extension (“invasion”) of the tumor into normal tissues.

Increasing Tissue Permeability and the Formation of New Blood Vessels (Angiogenesis).
The values of pHₑ measured (the pH of the immediate environment of the tumor); vascularization, angiogenesis and surrounding tissue permeability correlate with invasiveness and metastasis. Low pHₑ makes tumor cell lines more metastatic.
The energy metabolism of tumor cells being acidic is uniquely different from normal healthy viable cells and provides a electro physical basis for selective destruction of stages of advancement and all varieties of neoplasm's that may lead to cancers as well as a wide variety of cancerous tumors.
Cancerous viability is dependent on an acidic micro-environment. This is due in part to their aberrant energy metabolism which produces lactic acid and carbonic acid and in part to incomplete vascularization, which causes insufficient oxygen supply (hypoxia).
The common denominator for virtually all tumors is a reduced pH at the tumor's edge. The tumors pHₑ (micro environment) ranges from 5.5 to 7.2 with an optimum growth rate occurring at a pH of 6.6 to 6.9.
Definitions: Acidity and alkalinity are measured by pH which is defined as the negative logarithm of the hydrogen ion activity: pH=−log (H). The parameter pHₑ is the pH on the exterior and pHᵢ is the pH on the interior of the cell, as compared to systemic pH, which is the overall pH of the biological system.
The pH within tumor cells (pHᵢ) is similar to (or even more alkaline than) the pH of normal tissue cells. The pH of the micro-environment of the tumor (pHₑ), however, is more acidic than that of normal tissues. It should be noted that the term “tumor micro-environment” refers to both the non-cellular area within the tumor and the area directly outside the tumorous tissue and does not pertain to the intracelluar [sic] compartment of the cancer cell itself.
Tumors tend to be both hypoxic and acidic. Chronically hypoxic tissues are going to be (i.e. are always) acidic, whereas transiently hypoxic tissues may be acidic. The more central part of the tumor is hypoxic, the exterior is transiently hypoxic.
Cancers exhibiting the lowest pHₑ values are more acidic and more aggressive and hostile to the surrounding normal healthy cells and more likely to be fatal to the patient. Metastasis is responsible for nearly 90% of cancer deaths. Low pHₑ promotes persistent antigenic and metastic signaling, metastatic spread of cancer and neovascularization (including angiogenesis, enhancing blood flow to the tumor mass). Low pHₑ decreases the efficacy of the immune response to cancer cells. An acidic hypoxic micro-environment causes genomic instability, and increased resistance to conventional cancer treatment procedures (e.g., drugs, radiation).
There are other important consequences of aberrant energy metabolism. As compared to healthy cells, cancer cells have a lower energy charge (ATP (ADP+Pᵢ)). Additionally, all varieties of cancer cells typically have cellular distributions of ions that are different from normal healthy cells. Neoplastic and cancer cells usually contain excess internal sodium and grossly excess internal calcium, often with a deficiency in internal potassium. Cancer cells have ion fluxes across their membranes that are different from normal cells (e.g. increased H⁺ efflux). Cancer cells invariably have membrane electrical potentials (inside relative to outside) that are less electro-negative than normal cells. Aberrant ion concentrations such as high internal sodium or high internal calcium can induce apoptosis and or can modify the recognition of the cancer cell by the immune system.
Development of cancer involves a competition between the growth of neoplastic cells and their destruction by immunological processes. The genetic changes accompanying carcinogenesis have attracted great interest and much is known about the molecular mechanisms involved. Such changes are a prerequisite to the development of the malignant disease, but are not sufficient by themselves to overcome the immune defenses. Thus, cancer can be treated by therapies that potentiate the proper functioning of defenses such as immune response and apoptosis, so cancer propagation is shifted to promote cancer elimination.
The method and formula described in the invention have several related effects on development of the cancer micro-environment, both resulting from the same internal dynamics. First, the formula interferes with the hypoxic acidic energy metabolism of the cancer cells. This effect renders the cells less able to supply the energy required for the rapid proliferation typical of cancer cells resulting in a reduction or elimination in the viability zone of the cancer cells. Secondarily, the formula reduces acidification (both systemically and in the tumor micro-environment) and increases oxygenation, eliminating the adverse effects caused by acidic hypoxia.
4. Stem cell therapy involves the use of both autologous and (matched) heterologous bone marrow-derived cells for replacing the immune cell population in various types of leukemia and lymphoma. This therapy requires extreme safety measures and is highly stressful for patients. In addition, it is costly and limited to a small number of malignant diseases.
5. Immunotherapy employ several forms of immune cells isolated from patients blood (e.g. dendritic cells, lymphokine activated killer cells) which, after in vitro stimulation with tumor antigens or immune modulators, are re introduced to the patient. A limitation of the immunotherapeutic approach is the limited number of tumor types that have successfully been treated (e.g. melanoma, kidney tumors), the expensive and complex procedure and the limited success rates.

In addition to the enhanced metabolism, cancer cells also differ in their undesired hyperproliferation, i.e., their propensity, to avoid or overcome normal restraints on growth and division. Loss of growth control mechanisms leads the neoplastic cells to acquire unlimited replicative ability and to evade elimination, growth arrest, and senescence by tumor suppressors. In general, tumor suppressor genes block the transformation of normal cells to cancerous cells. As part of cancer development, at least some of these tumor suppressor genes must be eliminated or inactivated. One class of suppressor genes that is down-regulated relates to genes inducing apoptosis and other types of programmed cell death. Viruses, such as the flu virus, e.g., through PSB1-F2, can retilt the growth control to restore these constraints and allow natural elimination of the cells.

Regardless of the cell type originating the cancer, all cancer cells will present an increased uptake of nutrient building blocks into the cell, increased use of the nutrients (reactants) in various chemical reactions to make increased products. The products will include products useful for sustaining the cell and by-products such as waste chemicals and heat. While there are some common chemical waste products of metabolism, one ubiquitous product (since in general metabolism is exothermic) is an increased heat output.

Since cancer cells produce more heat than surrounding cells, increased temperature is a marker that can be used to identify and target these cells. While monitoring local temperature is not essential for all means of attacking cancer metabolism, heat can serve as a trigger or signal activating or making available an anti-cancer therapy. The cells essentially light-up or self-identify though their cancer adapted hypermetabolisms. Many physical or chemical tools that measure or monitor temperature are available to identify the cells or zones of cells with cancer associated hypermetabolic states. On a micro- or nano scale, electronic and/or chemical sensors can be made to accumulate at locations or at cell membranes that are responsible for characteristics such as increased temperature and decreased pH. Using specific characteristics of the hyperproliferating cancer cells allows these cells to be segregated from normally metabolizing cells and tissues.

Many physical or chemical tools that measure or monitor temperature are available to identify the cells or zones of cells with cancer associated hypermetabolic states. On a micro- or nano scale, electronic and/or chemical sensors can be made to accumulate at locations or at cell membranes that are responsible for characteristics such as increased temperature and decreased pH. Using specific characteristics of the hyperproliferating cancer cells allows these cells to be segregated from normally metabolizing cells and tissues. By localizing with the targeted cells the cell or zone of cells chemical or physical sensor compounds or components can isolate the targeted cells from healthy tissue cells and instigate one or more of several natural paths of these cells to their growth arrest and cell death. The isolated cells may be restrained by many possible interventions including, but not limited to: nutrient deprivation, membrane disruption, viral infection, mitochondrial autophagy, mitotic arrest, apoptosis stimulation, transcription alteration or cessation, interference RNA, etc. The innate immune system has evolved to include these and other control tools.

Nanoparticles can be mostly physical in their action, may include chemical elements to aid in sensing or for delivery and may even transport biologic cargo(es) depending on the whims of the nanoparticles creator(s).

Several forms of nano-particles are products of nature. Many or even most cell types are known to shed nano-sized vesicles formed by the inward budding of cellular compartments. These 40-100 nm sized known as multivesicular endosomes (mVE) fuse with the plasma membrane whereupon these cytoplasmic sourced vesicles are released as exosomes, capable of vascular or diffusive deliver to remote cells and tissues. When bound to a receptive target cell exosomes have been shown to influence diverse aspects of the cell's functions and physiology. The exosome's destiny is usually determined by its binding to cell receptors complementing specific ligands on the exosome surface. Exosomes can enter target cells through a target cell's endocytic pathway and/or through fusion with the target cell's cytoplasmic membrane. Exosome membrane can thus contribute lipids including lipid rafts and other structural components to the receptor cell or lipid membrane if the exosome has bound a non-cellular structure to that structure's external surface. Exosome internal contents are delivered directly into the recipient, e.g., a recipient cell's cytoplasm.

A similar cell derived structure may bud directly off the cytoplasmic membrane. These structures are called ectosomes, shed vesicles, or microvesicles. Such natural nano-particles are known couriers of bio-active proteins, inhibitory or productive RNAs, and reactive oxygen source material or reactive oxygens themselves.

Exosomes and ectosomes, shed vesicles, microvesicles and the like can be selectively produced, e.g., engineered in their outcome, through culturing and selectively culling or selectively proliferating one or more cell lineage to produce product with desired ligand binding characteristics, select membranous activities and/or preferred intraparticle contents for delivery to the chosen target. Lipids, proteins, and diverse nucleic acids including mRNAs, microRNAs (miRNAs), and other non-coding RNAs (ncRNAs) have been documented in the membrane or lumen of these particles. Exosomal RNAs can be taken up, for example, by neighboring cells or more distant cells when the nanoparticles enter circulation where they may subsequently modulate activities in the recipient cell.

The nanoparticle may target one or more membranous protein that acts a receptor for a ligand on the nanoparticle surface and/or through selective culturing or genetic engineering be equipped with pH seeking, heat seeking, high MHC expressing cells, etc. These nanoparticles, like other vectors or couriers that might deliver effective cell disabling or immune system activating components are available alternates for disabling a target cells ability to reproduce and survive natural clean-up operations in the organism.

Another form of natural or naturally derived nanoparticle can be obtained from selectively cultured or engineered viruses. Viruses can self-propagate as virions and can have varied structures for propagating their genetic materials. Viruses may be single stranded or double stranded. The genetic material may be DNA or RNA in all combinations. A virion or propagating viral particle may be a single or double stranded RNA (picornaviruses, togaviruses, orthomyxoviruses, rhabdoviruses, retroviruses or reoviruses, birnaviruses, respectively), a single or double stranded DNA (parvoviruses, annelloviruses, circoviruses or adenoviruses, herpesviruses, poxviruses, papoviruses, respectively). Viruses may comprise a single nucleic acid strand encoding all the viral genes or may be compilations of multiple nucleic acid molecules. For example, double stranded RNA viruses generally comprise one gene per RNA, while influenza As comprise eight individual RNA strands.

The orthomyxoviruses are exemplary as our common, but sometimes deadly flu virus. Influenzas A, B and C infect many warm-blooded vertebrates including humans and birds. Genera D viruses have been observed in farmed animals, but not yet in humans. Subtypes of each of genera A, B and C will infect the human and other mammalian organisms. Notable subtypes of A include, but are not limited to: H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, H10N7, etc. These nanoparticles may appear more spherical or more rodlike in shape and are somewhat larger (50-120 nm spheres) than exosomal particles or as thin as 20 nm to as long as several hundred nm when rodshaped.

Orthomyxoviruses or flu viruses may undergo slow change through small genetic changes passed down to daughter generations, or abruptly, through a process called “reassortment” where larger genetic segments swap between viral strains to create a new viral entity. Change is inherent in viral replication since each genome is independently polymerized and viruses have no capacity to correct misreads during duplication. Severe misreads simply cannot promulgate another generation either because their genes or gene products are nonfunctioning or they are outcompeted or easily identified and eliminated by the host immune defenses.

A more rapid change occurs in viruses comprising multiple nucleic acids, for example when one or more of the flu viruses' eight distinct nucleic acids swap between viruses. Both slow (genome misread) and abrupt (gene swap) changes can be useful for creating engineered or selectively cultured flu or flu-like nanoparticles. For example, a flu virus can be selected for high salt, high temperature, specific receptors, etc. by repeated culturing under selected conditions where the cultured virus essentially self-selects its genetic adaptations. Or one or more short or longer gene segments can be spliced into one of the genes encoding a complimentary protein ligand to the desired cell receptor. Enveloped viruses, since they bud from the host cell carrying the components of the host cell plasma membrane allow culture conditions, e.g., host cell chosen, temperature, cholesterol, phospholipids, ethanolamine and other membrane partitioning lipophilic and amphipathic molecules to determine viral constitution, especially of the envelope and thereby exert non-genetic control on its binding and melding with target cells. Culture cells and culture conditions are thus important and valid tools for selection and growth of the vector virus.

Co-infection of a cell, in the lab or in an organism, with two influenza viruses from different origins (e.g. avian and human), can result in mixing of the RNA segments from the two viruses and formation of a new virus with an altered genetic make-up. Such swapping of gene segments between viruses, i.e., genetic reassortment, is one mechanism by which new influenza viruses with pandemic potential may arise, but also a mechanism useful for engineering and selecting viral particles with desired traits.

For example, an H5N1 bird flu has been engineered (or modified) to infect humans. A surrogate mammalian species served as the culture medium for the bird flu which rapidly adapted to increase its proliferative abilities—by achieving airborne transmission capability. The relevant mutations were then sequenced providing a tool for engineering this trait into other virus species or subtypes. Such manipulations are common selection and engineering tools that might be used for optimization, in some instances merely routine optimization of infective virions, especially for example in phage viruses. Normal cell chaperones can be augmented in engineered culture cells to provide an efficient tool for assisted engineering of viral vectors with desired target cells and courier traits.

Influenza viruses A and B infections induce distinct apoptosis profiles. Apoptosis induced during influenza virus infection is a major contributing factor to symptoms including cell death and tissue damage. Influenza B induces an immediate apoptotic response favoring a caspace 8 pathway while influenza A favors the caspace 9 path to apoptosis. Either or both pathways may be turned on to induce cell death. The similarities in structure and genetic components of influenzas A and B can be used to form hybrids, e.g., by swapping one of the RNAs or by re-engineering a code segment on one or more of the RNAs to mimic the RNA of the other type. Influenza C generally is considered less virulent, often infecting without apparent symptoms. However, some influenza C strains may cause typically flu symptoms. Generally the course of infection is prolonged with apoptosis not always a terminal event. Accordingly, an influenza C can persist in the body for more extended periods and preferentially avoid an early adaptive immunity response.

Any available targeting or delivery means known in the art can be used. For example, a viral particle can be engineered to deliver a therapy to the targeted cell's interior. In the example of a reovirus which infects cells that express an activated ras oncogene, the cell is rendered more prone to infection by the virus since the activated Ras system deactivates antiviral defenses the cell would normally use to prevent reovirus infection. An engineered retrovirus, like a reovirus, or other vector known in the art is therefore a viable courier for a variety of therapeutic strategies to modulate intracellular metabolism especially when anti-viral defenses are compromised as often occurs when a cell ramps up its proliferative capacity.

Viral re-engineering has been a niche but is now a growing art. For example, Asokan et al, Nature biotechnology, volume 28: 1, Jan. 2, 2010, 79-82, teaches reengineering the receptor ligand of adeno-associated virus, with special emphasis on a basic [non-acidic]hexapeptide stretch at positions 585-590. (Charge and/or polarity of a peptide segment correlates positively with its availability for binding.) The engineered adeno-associated virus is defective in replication, requiring coinfection with another virus such as adenovirus, HSV, etc.
Madigan and Asokan, Current Opinion in Virology, Volume 18, June 2016, Pages 89-96 summarizes progress in engineering adeno-associated viral binding character. The glycan surface having been mapped, with multiple serotypes identified, isolated and characterized, bases for selecting optimal adeno-associated vectors is well-developed: “A thorough structural understanding of AAV capsid glycan interactions has enabled rational manipulation of glycan footprints on the AAV capsid surface. This re-engineering approach has yielded novel, synthetic AAV strains with potential applications in therapeutic gene transfer. Specifically, structure-inspired design has been utilized to abrogate capsid binding to glycan receptors, alter binding affinity, and more recently engineer orthogonal glycan receptor interactions.”

Multiple exemplary re-engineering successes are briefly mentioned in the paper along with a summary statement:
“A thorough structural understanding of AAV capsid glycan interactions has enabled rational manipulation of glycan footprints on the AAV capsid surface. This re-engineering approach has yielded novel, synthetic AAV strains with potential applications in therapeutic gene transfer. Specifically, structure-inspired design has been utilized to abrogate capsid binding to glycan receptors, alter binding affinity, and more recently engineer orthogonal glycan receptor interactions.” In this and other peer reviewed papers the adeno-associated virus is set forth as an advantageous candidate for vector re-engineering.

In the case of virus, strains of vaccinia virus, herpes virus, vesicular stomatitis virus, senaca virus, Semliki Forest virus, ECHO or REGVIR virus, and monstrously attenuated polio virus have been similarly tested and characterized in cancer cells or in animals or in humans with cancers for their inherent cell killing effects, primarily targeted at cancers.

For best efficiency the couriers will preferably transport a molecule whose effects are multiplied at or in the cell. For example, the courier may carry: RNAi with downstream effects on one or more of the cell's pathways, transcription factors, methylation factors, demethylation factors, an engineering cassette such as used in CRISPR/cas, a plasmid that can infect mitochondria, a ligand that opens a pore in an organelle such as the nuclear membrane or mitochondrial membrane, packets that increase expression of a protein or group of proteins to favor or disfavor one or more metabolic pathways (such as the electron transport pathway of mitochondria) to induce apoptosis, a cytokine, mitochondrial fusion or fission modulators, anti-apoptotic or pro-apoptotic compounds such as Bcl or Bad, etc.

Antisense RNA was recognized over 30 years ago as a means for suppressing synthesis from a complementary mRNA. However, the early attempts in using these to suppress expression showed unacceptable off-target effects. Improvements including double stranded RNAs have been recognized to have near universal effect in most cells of multicellular organisms and as such can provide a focal mechanistic system for the regulation of mRNA function. Many derivations are known in the art and are not repeated here. Antisense RNA incorporated into an engineered virus may specifically modulate one or more of the induced cellular proteins or may more generally modulate effects of one of the viral genes. For example, imbalancing production of a viral gene can reduce the virus' ant-immune effects.

Viruses naturally function by vectoring genetic material into cells they co-opt to produce more viral particles. Several viral genuses have had members engineered and used for treating cells. Viral particles of this invention may be selected for increased immunogenicity to activate the organism's immune system(s) to respond to the targeted site or may be selected for delayed or suppressed immunogenicity to encourage self-propagation of the sensor(s).

One genus is lentiviruses. Lentiviruses are a genus of viruses of the Orthoretrovirinae subfamily within the Retroviridae family. Members of this genus include pathogens of bovine, equine, feline, ovine, and primate receptor targets. Lentiviruses are enveloped viral particles that bud from an infected cell's plasma membrane in like fashion to influenza. Viral particles are 80 to 120 nm in diameter, containing a single-stranded 9.2-kb RNA genome and several structural proteins, including the matrix, capsid, nucleocapsid, envelope, and reverse transcriptase enzymes. Lentivectors feature efficient transduction of especially nondividing cells, minimal natural anti-vector immunity in animal hosts, and a low potential for genotoxicity resulting from insertional mutagenesis. Several modifications of the lentivector have improved their safety profile and ability to elicit a strong immune response. Viral particles bind to their target cell through the targeted cell's receptor and the virus's envelope glycoprotein. The particle fuses with the plasma membrane releasing the genomic RNA into the cell's cytoplasm where it is reverse transcribed to double stranded DNA on its path to incorporation in a host chromosome. Lentivirus genetic material is available for clipping and excising for incorporation into other virus genes, gene motifs, recognition sequences, etc.

In the 1980s retroviral particles were used to deliver therapeutic genes. Since these experiments, issues of viral particle instability, inability to transduce non-dividing cells and low titers have been addressed, e.g., by using engineered lentiviruses. Further engineering efforts including, but not limited to: elimination of viral genes for Vpr, Vif, Nef, and Vpu; replacing the Tat and 5′LTR with a constitutive promoter and moving Rev onto a different plasmid have improved safety and efficiency. Additional engineered features include, but are not limited to: adding woodchuck hepatitis B posttranscriptional regulatory element (WPRE) to improve gene expression; deleting the U3 region of the 30 LTR to generate self-inactivating transfer vectors (SINs); and including a triple-helix signal (TRIP) to improve nuclear import.

Specificity for host cells is engineered by modifying envelope proteins or transgene expression promoters. Vesicular stomatitis protein is one example for broadening the host repertoire. This or other stand-in gene can be engineered for pH and/or temperature selectivity. Such engineered lentiparticles have been used to vaccinate an organism and to induce cell suicide in targeted cells. Since the lentiparticle fuses with the plasma membrane such particles are suitable vectors for introducing various molecules including, but not limited to: siRNA, microRNA, snoRNA, lincRNA, a ribozyme, piRNA, double stranded and long double stranded ncRNA.

Engineering is an arbitrary term that may include targeted mutagenesis, selection mutagenesis, motif swapping, gene swapping, capsule or envelope substitution, etc. A virus, for example, an RNA virus, may be mutated to incorporate a sequence from another virus and possibly packaged in a coating co-produced during viral replication with a DNA virus. The viral type name may thus be arbitrarily based on the viral component relevant to a desired, selected, engineered, mutated, etc., activity.

Singly enveloped particles directly fuse with targeted membranes to release particle contents into the host cell cytoplasm. In a doubly enveloped format the particle is engulfed in an endocytotic process and the low pH cleaves the outer envelope allowing the inner envelope to fuse with the endosome membrane and release contents to the cytoplasm. Vaccinia can be engineered for selective, e.g., heat sensitive lipid envelope, pH sensitive envelope, selective lipid content etc. By selecting the threshold energy for fusion through propagating cell selection and/or engineering, vaccinia can be engineered for wider or narrower selectivity.

Genetic engineering tools can recognize specific mutations and when coordinated with an endonuclease can remove or edit identified genetic abnormalities. Systems such as CRISPR have recognized ability to distinguish methylated from non-methylated bases in genetic sequence.

Gene editing processes are continually being improved. To date they have improved precision and specificity and become acceptable in practice. An example of a recent summary of CRISPR technology appears in US patent Application 20170035860.
Gene editing technologies: Recent developments of technologies to permanently alter the human genome and to introduce site-specific genome modifications in disease relevant genes lay the foundation for therapeutic applications in CNS disorders such as Parkinson's disease (PD) or Alzheimer disease (AD). These technologies are now commonly known as “genome editing.” Current gene editing technologies comprise zinc-finger nucleases (ZFN), TAL effector nucleases (TALEN), and clustered regularly interspaced short palindromic repeats (CRISPR)/CRISPR-associated (Cas) system or a combination of nucleases (e.g. mutated Cas9 with Fokl) (Tsai, S. Q., Wyvekens, N., Khayter, C., Foden, J. A., Thapar, V., Reyon, D., Goodwin, M. J., Aryee, M. J., and Joung, J. K. (2014). Dimeric CRISPR RNA-guided Fold nucleases for highly specific genome editing. Nature biotechnology 32, 569-576.)) All three technologies create site-specific double-strand breaks. The imprecise repair of a double strand break by non-homologous end joining (NHEJ) has been used to attempt targeted gene alteration (nucleotide insertion, nucleotide deletion, and/or nucleotide substitution mutation). A double-strand break increases the frequency of homologous recombination (HR) at the targeted locus by 1,000 fold, an event that introduces homologous sequence at a target site, such as from a donor DNA fragment. Another approach to minimize off-target effects is to only introduce single strand breaks or nicks using Cas9 nickase (Chen et al., 2014; Fauser et al., 2014; Rong et al., 2014; Shen et al., 2014).
The CRISPR/Cas9 nuclease system can be targeted to specific genomic sites by complexing with a synthetic guide RNA (sgRNA) that hybridizes a 20-nucleotide DNA sequence (protospacer) immediately preceding an NGG motif (PAM, or protospacer-adjacent motif) recognized by Cas9. CRISPR-Cas9 nuclease generates double-strand breaks at defined genomic locations that are usually repaired by non-homologous end-joining (NHEJ). This process is error-prone and results in frameshift mutation that leads to knock-out alleles of genes and dysfunctional proteins (Gilbert et al., 2013; Heintze et al., 2013; Jinek et al., 2012). Studies on off-target effects of CRISPR show high specificity of editing by next-generation sequencing approaches (Smith et al., 2014; Veres et al., 2014) (FIG. 1, panel 1).
Other applications for heart disease, HIV, and Rett syndrome have been described. (Ding et al., 2014; Swiech et al., 2014; Tebas et al., 2014). For heart disease, permanent alteration of a gene called PCSK9 using CRIPR technology reduces blood cholesterol levels in mice (Ding et al., 2014). This approach was based on the observation that individuals with naturally occurring loss-of-function PCSK9 mutations experience reduced blood low-density lipoprotein cholesterol (LDL-C) levels and protection against cardiovascular disease (Ding et al., 2014). A second example for the feasibility of this approach is HIV. Individuals carrying the inherited Delta 32 mutation in the C—C chemokine receptor type 5, also known as CCR5 or CD195 are resistant to HIV-1 infection. Gene modification in CD4 T cells were tested in a safety trial of 12 patients and has shown a significant down-regulation of CCR5 in human (Tebas et al., 2014). Another recent study showed the successful use of CRISPR/Cas9 technology in CNS in a mouse model for the editing of the methyl-binding protein 2 (MecP2) gene. Mutation in this gene causes Rett syndrome, a condition in young children—mostly girls—with mental retardation and failure to thrive. In this approach an adeno-associated virus (AAV) was used as the delivery vehicle for the Cas9 enzyme in vivo. Overall, 75% transfection efficiency was described with a high targeting efficiency that almost completely abolished the expression of MecP2 protein and functionally altered that arborization of the neurons similar to what has been described for Rett syndrome (Swiech et al., 2014). This shows the proof of concept that gene editing using CRISPR/Cas9 technology is achievable in the adult brain in vivo.
Despite reports in the literature describing the use of genetic editing techniques, none have been described or suggested for genes associated with neurodegenerative disorders. A strong need continues to exist in the medical arts for a method for treating and/or inhibiting diseases associated with neurodegenerative disorders, such as materials and techniques useful for the treatment of Parkinson's Disease.

### SUMMARY OF THE INVENTION

In a general and overall sense, the present invention provides for the arrest and/or prevention of neurodegeneration associated with neurodegenerative disease in vivo. In some embodiments, arrest and/or prevention of neurodegeneration is accomplished using gene editing methodologies and molecular tools to manipulate specific gene(s) and/or gene regulatory elements, to provide a modification of the gene and/or genomic regions associated with neurodegeneration and neurodegenerative disease, such as Parkinson's Disease.
In some aspects, the present invention provides a method of treating a neurological deficit associated with neuropathological disease comprising administering a genetically engineered vector comprising a gene for a nuclease and a promoter for the nuclease, as well as an appropriate molecular “guide” into a cell. Following the administration, the vector facilitates an expression of a molecular component that alters a gene in the cell or expression of a targeted gene associated with the neuropathology in the cell. The affected gene would be implicated in an etiology of the neurological deficit.
In other embodiments, a medical composition for treating a neurological deficit in a patient is provided. The medical composition includes a nuclease that introduces double strand break in a gene implicated a neurological deficit, a guide RNA that targets a gene implicated in neurological disease, and a delivery system that delivers the nuclease and guide RNA to a cell.
For purposes of the description of the present invention, the term “modification of gene and/or genomic region” may be interpreted to include one or more of the following events (FIG. 1):
a) Targeted introduction of a double-strand break by a composition disclosed, resulting in targeted alterations (random mutations e.g. insertions, deletions and/or substitution mutations) in one or more exons of one or more genes. This modification in some embodiments provides a permanent mutation in a cell or population of cells having the modified gene.
b) Targeted binding of non-functional mutant Cas9 to non-coding regions (e.g. promoters, evolutionary conserved functional regions, enhancer or repressor elements). Binding is induced by compositions disclosed. Sterical hindrance of binding of other proteins (e.g. transcription factors, polymerases or other proteins involved in transcription) may also result as a consequence of binding.
1. CRISPR sgRNA introduces small insertions or deletions through non-homologous end joining (NHEJ), in general several nucleotides, rarely larger fragments (Swiech et al., 2014).
2. Homology-directed repair (HDR) to correct point mutations by introducing a non-natural, but partially homologous template.
3. Double Genome editing of splice-sites or splicing related non-coding elements to eliminate certain gene regions, e.g. exon 5 of SNCA gene.
4. Double Genome editing of non-coding or intronic gene regions to eliminate regulatory elements that increase or decrease gene expression, e.g. D6 or 112 regulatory region in SNCA gene.
5. sgRNA guides mutant Cas9 to physically inhibit binding of transcription factors in promoter region,
6. sgRNA guides mutant Cas9 to physically inhibit binding of transcription factors in regulatory regions or intronically.
Gene editing or modification can be achieved by use of any variety of techniques, including zinc-finger nuclease (ZFN) or TAL effector nuclease (TALEN) technologies or by use of clustered, regularly interspaced, short palindromic repeat (CRIPSR)/Cas9 technologies or through the use of a catalytically inactive programmable RNA-dependent DNA binding protein (dCas9) fused to VP16 tetramer activation domain, or a Krueppel-associated box (KRAB) repressor domain, or any variety of related nucleases employed for gene editing. These can be seen as existing tools to sever the genomic region in question.
The tools mentioned above, are general in their application. Aspects of the present methods and compositions provide the design of custom CRISPR single-guide RNA (sgRNA) sequences specific for coding gene regions and regulatory sequences in genes implicated in neurodegeneration. In this manner, an exact genomic location for precise gene alteration in humans may be accomplished, with a resulting improvement and/or elimination of a neurodegenerative disorder pathology or symptom.
Additional patents and patent applications, for example, US application no. 20170015994 evidence the utility, feasibility and enablement of gene editing processes with high specificities are well known and accepted in the art.

### Vitamin D

The natural immune response is supported by other natural systems in the body. Accordingly, in several embodiments it will be advantageous to up-regulate or down-regulate one or more supportive pathways. One example relates to calcium homeostasis, in particular Vitamin D. For example U.S. Pat. No. 9,149,528 granted Oct. 6, 2015 to William A. McHale and Dale G. Brown recognizes the support that vitamin D provides to controlling the immune system response:
Vitamin D is known as a key player in calcium homeostasis and electrolyte and blood pressure regulation. Recently, important progress has been made in understanding how the noncanonical activities of Vitamin D influence the pathogenesis and prevention of human disease. Vitamin D and VDR are directly involved in T cell antigen receptor signaling. The involvement of vitamin D/VDR in anti-inflammation and anti-infection represents a newly identified and highly significant activity for VDR. Studies have indicated that the dysregulation of VDR may lead to exaggerated inflammatory responses, raising the possibility of defects in vitamin D and VDR signaling transduction may be linked to bacterial infection and chronic inflammation including periodontitis.
Overall, the effects of 1,25(OH)₂D₃ on the immune system include: modulating the TCR, decreasing Th1/Th17CD4+ T cells and cytokines, increasing regulatory T cells, downregulating T cell-driven production and inhibiting DC differentiation.
Consistent with its anti-inflammatory role, 1,25(OH)₂D₃ downregulates the expression of many proinflammatory cytokines, such as IL-1, IL-6, IL-8 and TNF-α, in a variety of cell types. Immune cells, including macrophages, DCs and activated T cells, express the intracellular VDR and are responsive to 1,25(OH)₂D₃.
Epidemiological studies suggest that low vitamin D levels may increase the risk or severity of respiratory viral infections. One study examined the effect of vitamin D on respiratory syncytial virus (RSV)-infected human airway epithelial cells. Airway epithelium converts 25-hydroxyvitamin D3 (storage form) to 1,25-dihydroxyvitamin D3 (active form). Active vitamin D generated locally in tissues, is important for the non-skeletal actions of vitamin D, including its effects on immune responses. It was found that vitamin D induces IkBα, an NF-kB inhibitor, in airway epithelium and decreases RSV induction of NF-kB-driven genes such as IFN-β and CXCL10. It was also found that exposing airway epithelial cells to vitamin D reduced induction of IFN-stimulated proteins with important antiviral activity (e.g., myxovirus resistance A and IFN-stimulated protein of 15 kDa). In contract to RSV-induced gene expression, vitamin D had no effect on IFN signaling, and isolated IFN induced gene expression. Inhibiting NF-kB with an adenovirus vector that expressed a nondegradable form of IkBa mimicked the effects of vitamin D. When the vitamin D receptor was silenced with small interfering RNA, the vitamin D effects were abolished. Most importantly it was found that, despite inducing IkBa and dampening chemokines and IFN-β, there was no increase in viral mRNA or protein or in viral replication.
Vitamin D is increasingly recognized as a pluripotent hormone with functions that extend beyond its classical role in calcium homeostasis. Rapidly growing evidence from epidemiological and basic research studies reveals that vitamin D can modulate immune responses. Vitamin D deficiency is highly prevalent and has been associated with both increased risk of several inflammatory diseases and susceptibility to infections, including periodontitis. The localized tissue-specific generation of active vitamin D is thought to be a key component of nonclassical vitamin D functions that are relied on by the supplement compositions of the invention. Previously published data has shown that normal lung epithelium constitutively converts 25-hydroxyvitamin D3 (storage form of vitamin D) to 1,25-dihydroxyvitamin D3(1,25D) (active form of vitamin D) and that the generation of active vitamin D is increased in the presence of viral infection.
The family of NF-kB transcriptional regulatory factors has a central role in coordinating the expression of a wide variety of genes that control immune responses. NF-kB proteins are present in the cytoplasm in association with 1 kBs. IkBs are phosphorylated by IkB kinase following cell stimulation, and they are targeted for destruction by the ubiquitin/proteasome degradation pathway. The degradation of IkB allows NF-kB proteins to translocate to the nucleus, bind to their DNA binding sites and activate a variety of genes. See: Sif Hansdottir, et. al., The Journal of Immunology. 2010; 184: 965-974.

The body's first line of the defense against pathogenic challenge the innate system occurs in an immediate and non-specific manner. This involves the complement system, antibacterial responses by neutrophils and macrophages, and incorporates antigen presentation to lymphocytic cells for the adaptive or acquired immune system. Vitamin D is involved in regulating various components of the innate immune system. CYP27B1 is the enzyme that activates vitamin D. Renal CYP27B1 is regulated by endocrine factors associated with calcium and phosphate homeostasis such as parathyroid hormone and fibroblast growth factor 23. But outside the kidney CYP27B1 is regulated differently.

Monocytes and macrophages are crucial members of the innate immune compartment, being able to sense the pathogen-associated molecular patterns (PAMPs) expressed by these pathogens. PRRs, such as toll-like receptors (TLRs), that are expressed, e.g., by monocytes. Monocytic TLR2/1 specifically inductes CYP27B1 and vitamin D receptor (VDR). Following activation both 1,25(OH)2D and 25OHD induced expression of LL37 in macrophages.

As a consequence, individuals with vitamin D-insufficiency (low serum 25OHD) will be less able to support monocyte induction of LL37, and may therefore present with compromised innate immunity including ability to mount an immune response to developing or developed cancer cells. While the present invention is not reliant on vitamin D levels, properly activated vitamin D, supplements and/or activation protocols may be used to enhance positive outcome.

Many different chemicals produced as part of the immune response can now be made in the laboratory. These include interferons, interleukin 2 and monoclonal antibodies now available ion the clinic. When relative terms, e.g., larger, more rapid, reduced, etc., are used in discussion or in claims they generally refer to the engineered or selected version in comparison to the isolated version serving as a source product for the engineering exercise.

Interferon alpha and interleukin 2 are known to act by boosting the immune response to help the body kill off cancer cells. Facilitating the body's natural defenses with devices such as an engineered virus as described in this application is thus a credible utility recognized to address an unmet need.

1. An engineered vector carrying a message for inducing death in a hyperproliferative cell of a homeothermic animal, said vector comprising i) a recognition component directing said vector to a cell whose temperature is greater than the temperature of surrounding tissue; ii) a recognition component directing said vector to a cell whose [H⁺] is greater than the [H⁺] of surrounding tissue; iii) a ligand for binding said hyperproliferative cell; and iv) a chemical messenger that facilitates apoptosis in said hyperproliferative cell wherein said hyperproliferative cell is selected from the group consisting of: a cancer cell and a precancerous cell.

2. The engineered vector of claim 1 wherein said homeothermic animal is selected from the group consisting of domesticated animals.

3. The engineered vector of claim 2 wherein said homeothermic animal is selected from the group consisting of: pets and zoo animals.

4. The engineered vector of claim 3 wherein said pets comprises a group selected from the group consisting of: canines and felines.

5. The vector of claim 1 wherein said chemical messenger comprises a virus.

6. The vector of claim 5 wherein said virus is selected from the group consisting of: picornaviruses, togaviruses, orthomyxoviruses, rhabdoviruses, retroviruses, reoviruses, birnaviruses, parvoviruses, annelloviruses, circoviruses, adenoviruses, herpesviruses, poxviruses and papoviruses.

7. The vector of claim 6 wherein said virus is selected from the group consisting of: orthomyxoviruses.

8. The vector of claim 7 wherein said virus is selected from the group consisting of: influenza A, influenza B and influenza C.

9. The vector of claim 1 wherein said chemical messenger comprises at least one engineered gene that operates in facilitating said apoptosis in said hyperproliferative cell.

10. The vector of claim 9 wherein said facilitating apoptosis comprises inducing at least one innate immune response in said hyperproliferative cell.

11. The engineered vector of claim 8 comprising an engineered influenza virus wherein said engineered influenza virus is engineered to reduce anti-apoptotic activity of viral infection in said at least one hyperproliferative cell.

12. The engineered vector of claim 8 wherein said engineered influenza virus is engineered to inhibit expression of at least one anti-apoptotic protein in said at least one hyperproliferative cell.

13. The engineered vector of claim 8 wherein said engineered influenza virus is engineered to support expression of at least one pro-apoptotic protein in said at least one hyperproliferative cell.

14. The engineered vector of claim 8 wherein said engineering comprises modifying sequence or availability of an RNA comprising a viral gene encoding a protein selected from the group consisting of: PB1-F2, NS1, M1, M2, HA, NP, NS2, NEP, PB1, PB2 and NA.

15. The engineered vector of claim 14 wherein PB1-F2 is engineered to support apoptosis.

16. The engineered vector of claim 14 wherein said engineering increases activity of a host cell component selected from the group consisting of: TLR3, TLR7, IRF7, MDA5, RIGI

17. The engineered vector of claim 14 wherein said engineering increases activity of a host cell component selected from the group consisting of: IFNB1, IL28A, IL29, IL28B, IFNW1, IFNA7, IFNA14, IFNA10, IFNA13, IFNA16, IFNA8, IFNA1, IFNG, IFNA2, and IFNA21.

18. The engineered vector of claim 14 wherein said engineered influenza virus is engineered to increase PB1-F2 expression.

19. The engineered vector of claim 14 wherein said engineered influenza virus is engineered to increase PB1-F2 delivery to mitochondria.

20. The engineered vector of claim 14 wherein said engineered influenza virus is engineered to increase NA expression.

21. The engineered vector of claim 14 wherein said engineering comprises modifying sequence of an RNA to enhance production of CpG-immunostimulatory oligonucleotides.

22. The engineered vector of claim 1 wherein facilitating apoptosis comprises inducing expression of at least one cytokine selected from the group consisting of: chemokines, interferons, interleukins, lymphokines and tumor necrosis factors.

23. The engineered vector of claim 10 wherein said innate immune response comprises inducing expression of at least one interferon.

24. The engineered vector of claim 8 wherein said innate immune response comprises activation of at least one caspace.

25. The engineered vector of claim 8 wherein said engineered influenza virus is engineered for increased reactivity with at least one mammalian TLR.

26. The engineered vector of 25 wherein said mammalian TLR comprises a feline or canine TLR.

27. The engineered vector of claim 25 wherein said at least one mammalian TLR is selected from the group consisting of: TLR2, TLR3, TLR4, TLR7 and TLR9.

28. The engineered vector of claim 8 wherein said influenza virus is selected from the group consisting of influenza As.

29. The engineered vector of claim 28 wherein said influenza A is selected from the group consisting of: H1N1, H1N2, H2N2, H3N1, H3N2, H3N8, H5N1, H5N2, H5N3, H5N8, H5N9, H7N1, H7N2, H7N3, H7N4, H7N7, H7N9, H9N2 and, H10N7.

30. The engineered vector of claim 5 wherein said engineered influenza virus is engineered for increased MHC class I presentation.

31. The engineered vector of claim 8 wherein said engineered influenza virus is engineered for more robust MHC class I presentation resulting in increased efficiency of apoptosis of the infected cell.

32. The engineered vector of claim 8 wherein said influenza is selected from the group consisting of influenza Cs with enhanced cytopathic effect.

33. The engineered vector of claim 8 wherein said engineering comprises targeted mutagenesis.

34. The engineered vector of claim 8 wherein said engineering comprises serial passaging.

35. The engineered vector of claim 1 as part of a preparation further comprising at least one apoptosis stimulant.

36. The engineered vector of claim 35 wherein said apoptosis stimulant enhances nitric oxide availability.

37. The engineered vector of claim 35 wherein said apoptosis stimulant is selected from the group consisting of: YCKVILTHRCY, GRVCLTLCSRLT, cannabidiol, kaempferol, URB937, Costunolide, TW-37, Epibrassinolide, 2-arachidonoylglycerol, 15-acetoxy Scirpenol, NSC 687852 (b-AP15), Cycloheximide, Bendamustine HCl, CFM 4, 7BIO, MPI-0441138, Citrinin, Destruxin B, (±)-Jasmonic Acid methyl ester, Psoralidin, JWH-015, ML-291, F16, Mitomycin C, Betulinic acid, BAM7, Kaempferol, Gambogic Acid, Apicidin, 2-Methoxyestradiol (2-MeOE2), Kaempferol, dexamethasone, 3,3′-Diindolylmethane, Brassinolide, Capsaicin, Triciribine Curcumin, Matrine, R1530, SMIP004, Trabectedin, 2,3,7, 8-tetrachlorodibenzo-p-dioxin, PM00104, Meisoindigo, 2,3-DCPE hydrochloride, Actinomycin D, Raltegravir potassium salt, C 75, Atractyloside Dipotassium Salt, CHM 1, Deguelin, Oncrasin 1, Streptozocin, Piperlongumine, FAAH inhibitors, Gambogic Acid, Linoleic Acid, PKC-412, Z3902, V9389, T7329, T2577, SRP5180, SRP5168, SRP5166, SRP5164, SRP4928, SRP3199, SRP3047, SRP3046, SML1908, SML1903, SML1843, SML1827, SML1823, SML1793, SML1765, SML1758, SML1745, SML1710, SML1707, SML1660, SML1637, SML1635, SML1601, SML1576, SML1533, SML1493, SML1492, SML1490, SML1464, SML1456, SML1372, SML1306, SML1302, SML1269, SML1263, SML1187, SML1156, SML1131, SML1016, SML1013, SML0991, SML0978, SML0963, SML0954, SML0953, SML0932, SML0907, SML0892, SML0821, SML0641, SML0623, SML0610, SML0580, SML0552, SML0521, SML0507, SML0433, SML0417, SML0404, SML0367, SML0363, SML0256, SML0188, SML0140, SML096, SML040, SMLOO031, SMB00431, SMB00418, SMB00388, S7451, S7448, R9156, R5030, R3530, PZ0115, P1499, P0103, P0069, N9162, N6287, M7888, K4394, 17160, 15159, H8787, H4663, G8171, G7923, G7548, F9428, E9661, E7781, E5411, E5286, E5161, E4660, D7446, D5817, C9369, C7744, C5865, C5492, C4992, C1244, BM0018, B8809, B5936, B5437, B3061, B0261, A8476, A4233 and A3105.

38. The preparation of claim 35 further comprising vitamin D or a vitamin D activator.

39. A method for inducing death in a hyperproliferative cell in a homeothermic animal, said method comprising selecting or engineering an influenza virus to increase binding compared with a common influenza virus at an increased temperature and selecting or engineering said influenza virus to increase binding compared with a common influenza virus at an increased H⁺ concentration, delivering said engineered influenza virus to said homeothermic animal, and stimulating an auto-immune response in said at least one hyperproliferative cell leading to its death.

40. The method of claim 39 wherein said engineered influenza virus facilitates apoptosis in said at least one hyperproliferative cell.

41. The method of claim 39 wherein said engineered influenza virus is engineered to reduce anti-apoptotic activity of viral infection in said at least one hyperproliferative cell.

42. The method of claim 39 wherein said engineered influenza virus is engineered to inhibit expression of at least one anti-apoptotic protein in said at least one hyperproliferative cell.

43. The method of claim 39 wherein said engineered influenza virus is engineered to support expression of at least one pro-apoptotic protein in said at least one hyperproliferative cell.

44. The method of claim 39 comprising modifying sequence or availability of a viral gene encoding a protein selected from the group consisting of: PB1-F2, NS1, M1, M2, HA, NP, NS2, NEP, PB1, PB2 and NA.

45. The method of claim 44 wherein PB1-F2 is engineered to support apoptosis.

46. The method of claim 45 wherein said engineered influenza virus is engineered to increase PB1-F2 expression.

47. The method of claim 44 wherein said engineered influenza virus is engineered to increase PB1-F2 delivery to mitochondria.

48. The method of claim 44 wherein said engineered influenza virus is engineered to increase NA expression.

49. The method of claim 39 wherein said auto-immune response comprises inducing expression of at least one cytokine selected from the group consisting of: chemokines, interferons, interleukins, lymphokines and tumor necrosis factors.

50. The method of claim 39 wherein said auto-immune response comprises inducing expression of at least one interferon.

51. The method of claim 39 wherein said auto-immune response comprises activation of at least one caspace.

52. The method of claim 39 wherein said selecting or engineering comprises selecting cultures conditions that improve viral envelope melding with a plasma membrane of said hyperproliferative cell at an increased temperature. The method of claim 35 wherein said selecting or engineering comprises selecting cultures conditions that improve viral envelope melding with a plasma membrane of said hyperproliferative cell at an increased temperature.

53. The method of claim 39 wherein said selecting or engineering comprises selecting cultures conditions that improve viral envelope melding with a plasma membrane of said hyperproliferative cell at an increased [H⁺].

54. The method of claim 40 wherein said engineered influenza virus is engineered for increased reactivity with at least one mammalian TLR.

55. The method of claim 54 wherein said at least one mammalian TLR is selected from the group consisting of: TLR2, TLR3, TLR4, TLR7 and TLR9.

56. The method of claim 39 wherein said influenza virus is selected from the group consisting of: influenza A, influenza B and influenza C.

57. The method of claim 56 wherein said influenza A is selected from the group consisting of: H1N1, H1N2, H2N2, H3N1, H3N2, H3N8, H5N1, H5N2, H5N3, H5N8, H5N9, H7N1, H7N2, H7N3, H7N4, H7N7, H7N9, H9N2 and, H10N7.

58. The method of claim 39 wherein said engineered influenza virus is engineered for increased MHC class I presentation.

59. The method of claim 39 wherein said engineered influenza virus is engineered for more robust MHC class I presentation resulting in increased efficiency of apoptosis of the infected cell.

60. The method of claim 56 wherein said influenza C is selected with enhanced cytopathic effect.

61. The method of claim 39 further comprising enhancement of apoptosis using one or more apoptosis stimulant.

62. The method of claim 59 wherein said apoptosis stimulant is selected from the group consisting of: nitric oxide, UV light, oxygen stress, increased temperature.

63. The method of claim 59 wherein said apoptosis stimulant is selected from the group consisting of: YCKVILTHRCY, GRVCLTLCSRLT, cannabidiol, kaempferol, URB937, Costunolide, TW-37, Epibrassinolide, 2-arachidonoylglycerol, 15-acetoxy Scirpenol, NSC 687852 (b-AP15), Cycloheximide, Bendamustine HCl, CFM 4, 7BIO, MPI-0441138, Citrinin, Destruxin B, (±)-Jasmonic Acid methyl ester, Psoralidin, JWH-015, ML-291, F16, Mitomycin C, Betulinic acid, BAM7, Kaempferol, Gambogic Acid, Apicidin, 2-Methoxyestradiol (2-MeOE2), Kaempferol, dexamethasone, 3,3′-Diindolylmethane, Brassinolide, Capsaicin, Triciribine Curcumin, Matrine, R1530, SMIP004, Trabectedin, 2,3,7, 8-tetrachlorodibenzo-p-dioxin, PM00104, Meisoindigo, 2,3-DCPE hydrochloride, Actinomycin D, Raltegravir potassium salt, C 75, Atractyloside Dipotassium Salt, CHM 1, Deguelin, Oncrasin 1, Streptozocin, Piperlongumine, FAAH inhibitors, Gambogic Acid, Linoleic Acid, PKC-412, Z3902, V9389, T7329, T2577, SRP5180, SRP5168, SRP5166, SRP5164, SRP4928, SRP3199, SRP3047, SRP3046, SML1908, SML1903, SML1843, SML1827, SML1823, SML1793, SML1765, SML1758, SML1745, SML1710, SML1707, SML1660, SML1637, SML1635, SML1601, SML1576, SML1533, SML1493, SML1492, SML1490, SML1464, SML1456, SML1372, SML1306, SML1302, SML1269, SML1263, SML1187, SML1156, SML1131, SML1016, SML1013, SML0991, SML0978, SML0963, SML0954, SML0953, SML0932, SML0907, SML0892, SML0821, SML0641, SML0623, SML0610, SML0580, SML0552, SML0521, SML0507, SML0433, SML0417, SML0404, SML0367, SML0363, SML0256, SML0188, SML0140, SML096, SML040, SML031, SMB00431, SMB00418, SMB00388, S7451, S7448, R9156, R5030, R3530, PZ0115, P1499, P0103, P0069, N9162, N6287, M7888, K4394, 17160, 15159, H8787, H4663, G8171, G7923, G7548, F9428, E9661, E7781, E5411, E5286, E5161, E4660, D7446, D5817, C9369, C7744, C5865, C5492, C4992, C1244, BM0018, B8809, B5936, B5437, B3061, B0261, A8476, A4233 and A3105.

64. The method of claim 39 further comprising assessing vitamin D levels in said organism and supplementing said organism with vitamin D and/or activating vitamin D in said organism to at least vitamin D levels recommended by the FDA guidelines.

65. The method of claim 39 further comprising enhancing said organism's levels of activated vitamin D.

66. The method of claim 39 further comprising engineering said influenza virus to increase transcription of at least one of a cell's pro-apoptotic proteins.

67. The method of claim 39 wherein said influenza virus is selected or engineered to induce expression of at least one DD protein.

68. The method of claim 39 wherein said engineering comprises targeted mutagenesis.

69. The method of claim 39 wherein said engineering comprises serial passaging.
